SEQUENCE LISTING (2 sequences)

SEQ ID NO: 1 - protein, 1239 aa, SEQUENCE ID No. 1, POLG1 protein sequence
MSRLLWRKVAGATVGPGPVPAPGRWVSSSVPASDPSDGQRRRQQQQQQQQQQQQQPQQPQVLSSEGGQLRHNPLDIQMLSRGLHEQIFGQGGEMPGEAAVRRSVEHLQKHGLWGQPAVPLPDVELRLPPLYGDNLDQHFRLLAQKQSLPYLEAANLLLQAQLPPKPPAWAWAEGWTRYGPEGEAVPVAIPEERALVFDVEVCLAEGTCPTLAVAISPSAWYSWCSQRLVEERYSWTSQLSPADLIPLEVPTGASSPTQRDWQEQLVVGHNVSFDRAHIREQYLIQGSRMRFLDTMSMHMAISGLSSFQRSLWIAAKQGKHKVQPPTKQGQKSQRKARRGPAISSWDWLDISSVNSLAEVHRLYVGGPPLEKEPRELFVKGTMKDIRENFQDLMQYCAQDVWATHEVFQQQLPLFLERCPHPVTLAGMLEMGVSYLPVNQNWERYLAEAQGTYEELQREMKKSLMDLANDACQLLSGERYKEDPWLWDLEWDLQEFKQKKAKKVKKEPATASKLPIEGAGAPGDPMDQEDLGPCSEEEEFQQDVMARACLQKLKGTTELLPKRPQHLPGHPGWYRKLCPRLDDPAWTPGPSLLSLQMRVTPKLMALTWDGFPLHYSERHGWGYLVPGRRDNLAKLPTGTTLESAGVVCPYRAIESLYRKHCLEQGKQQLMPQEAGLAEEFLLTDNSAIWQTVEELDYLEVEAEAKMENLRAAVPGQPLALTARGGPKDTQPSYHHGNGPYNDVDIPGCWFFKLPHKDGNSCNVGSPFAKDFLPKMEDGTLQAGPGGASGPRALEINKMISFWRNAHKRISSQMVVWLPRSALPRAVIRHPDYDEEGLYGAILPQVVTAGTITRRAVEPTWLTASNARPDRVGSELKAMVQAPPGYTLVGADVDSQELWIAAVLGDAHFAGMHGCTAFGWMTLQGRKSRGTDLHSKTATTVGISREHAKIFNYGRIYGAGQPFAERLLMQFNHRLTQQEAAEKAQQMYAATKGLRWYRLSDEGEWLVRELNLPVDRTEGGWISLQDLRKVQRETARKSQWKKWEVVAERAWKGGTESEMFNKLESIATSDIPRTPVLGCCISRALEPSAVQEEFMTSRVNWVVQSSAVDYLHLMLVAMKWLFEEFAIDGRFCISIHDEVRYLVREEDRYRAALALQITNLLTRCMFAYKLGLNDLPQSVAFFSAVDIDRCLRKEVTMDCKTPSNPTGMERRYGIPQGEALDIYQIIELTKGSLEKRSQPGP

SEQ ID NO: 2 - DNA, 18478 bp, SEQUENCE ID No. 2, POLG1 genomic gene locus
gcggaccggccgggtggaggccacacgctaccccgaggctgcgtaggccgcgcgaagggggacgccgtgccgtgggcctggggtcgggggagcagcagaccgggaagcaccgtgaggaccgaggtttccggcggggtcggcggcggggaggccgggtcgctgagcgacggcgcggcccctccctctccagtcagggagcgaggcccggagcagggcggcggctagtcccagggcgcaccgcggcgcctctgccgggcgcaggcgggcggcggggcgcacgggggtggccgccgactcctcctgcaggacgctctcggccgggtgggccgtggtccgggtgtgggtgtgggtcccgggggacggcggcccaccctgcgggttcgaatccgggcgctggcacctctcgacgctaggcccgcgccggtcgcggtaatggcagccaccatttgccgagcgcttgccaagagcagggccgcacgacataggcgccctgtgtcccccagacagcagcccggtgtgacaggcagaatccgtaatcccattttacagaataggatatcagggcctaaggagctttgcccaaggtcacacagctcgagagagccagaagcggggttcaaaaccgcgtcgccctactccagatactgctctcttactcgctgccctcggcttccccacgtgggttcactgacgaagttgcgtggaccccggttcccccaggaggggtattgacgtttcccaagttttgaggcttaacggaaaatgcaactgaagcgcctggcacagtgttggggacgcagtaaatgctcaaggaatgatgattatggatacacctattacatatatggtaaaataacgctttatatcatctgtctcctttaggatttggggtggaaggcaggcatggtcaaacccatttcactgacaggagagcagagacaggacgtgtctctctccacgtcttccagccagtaaaagaagccaagctggagcccaaagccaggtgttctgactcccagcgtgggggtccctgcaccaaccatgagccgcctgctctggaggaaggtggccggcgccaccgtcgggccagggccggttccagctccggggcgctgggtctccagctccgtccccgcgtccgaccccagcgacgggcagcggcggcggcagcagcagcagcagcagcagcagcagcagcaacagcagcctcagcagccgcaagtgctatcctcggagggcgggcagctgcggcacaacccattggacatccagatgctctcgagagggctgcacgagcaaatcttcgggcaaggaggggagatgcctggcgaggccgcggtgcgccgcagcgtcgagcacctgcagaagcacgggctctgggggcagccagccgtgcccttgcccgacgtggagctgcgcctgccgcccctctacggggacaacctggaccagcacttccgcctcctggcccagaagcagagcctgccctacctggaggcggccaacttgctgttgcaggcccagctgcccccgaagcccccggcttgggcctgggcggagggctggacccggtacggccccgagggggaggccgtacccgtggccatccccgaggagcgggccctggtgttcgacgtggaggtctgcttggcagagggaacttgccccacattggcggtggccatatccccctcggcctggtaagtaggggcagggttggggacataagcaggcatgggggcccagcttaatagtttgtttcagtgaacattttctgaggtcctgttacgggctgggtgctcacgtagggagcgctgatgtgttgaattaggactagacccctgtttatgtgggactcactttctggtgggaagatcacaggcagtaagcaaatacccaagtaaatgtcaggcagtaaaggccacgcagagaatcacagtagagcgctgtacatgagaccttcgggaggccacttaagatcacggtgatttggtgcctttaccccctctcctaatagcgtcatgagaagttagtctgaaaagtcatttgaacagtgtttctatttggggagctattaattattttgggcggtagaaagctcccttttgtgggactgtcccaggcagtataggacatttagcatccccagcctttcccataaacgccagaccaacacccccccgcccccctgcccccgccggcaacgtttccagacgcccccttgaggtggcatctggttgaccacccctagttgagaaacattgcttccttcccccagccttccaagcaggcattttggtcccaaacaagtatatccaatctctcttttctttttaaataactttctaagtgctacccaagtttctttttcaaacaatgatggcagtactgtttctcccctttttttattcttcattccaggattaaaatactatttacaaccttaatgctttcaggcatggccagcaaaaaagttggcagtttctttattcctattggaagctacatctttgtaaagaaagctgcgaaatgttaaatatgcagttgaaaatggtgaaaacatggctaaatagataaggtaggcattaatggctgaaaagagcaaaactagatgattctgcattgattgagttccagttacaatgagaatcacactacttagaatatgtaacttgatggtcaaagtaaaggggaatatcggccatcatttgaaaagataaagtaggctttggtggctgaaagaaaattaggaaaccagtgacaagaaagatttgttttttgatctgtcggtcattttaggccaaattacctcaagtccccttttcttttctctttctccttctttctctctttttacctctcctttccctccctgtccttccctgctctgccctcattctcattccattcttgccagtggtactcggggcattgcttagttgacctgatggcagaagtcactgttaaggcctgggctcatgctgggaccttcctcctgggagtctgactggtgggtgggggtgggtgccacatggtgccctaatagcggtccactttgaacctgggcatgcccctgccccttagctgagtaacattaggtacctgaccagcccacaatttacaatgggaggagaagcggtagtcagctatgagcctcccacagggcagcttcttcccaaagggtgttggtaagggcttcggccatcaggctagagggacgtctctctggccatcagcatttttctaagattcacagtaaaactagtattaatggcatggatccctactcatcttaaatttggcttgtttctttttaatcactagtttataatatggcttcatgcacagctgcagagctgcatcttgacaccagtgtggctttttactgtaaccaaagttcctgttaccaccatggcctcaaagatttggcattctttagcctttttgtctgcgttgttttaagggctttgacatgctgaattaaaatgtgggggggtggggatttctttcagtcccttggcttattttcaccatttggagtatgagttcgattttgtcaggtttaaaactaggaacctctttttgctttctctttgaaagaagttagttttatgtgtgttgaatctgttgaggcagatactccctttttcccttccataaaggttgcaaggagctccttcgcagctgtgttgtccacacgtggcctcgtcactcactttgatgctgagtgggccttgattgtttagaataatctgtggcttgcaacaggcatttcctcagtggccattcccctacacctagccttgtggatcttgagcaaactgcagccttttcctgaatcagtgtcgggcccccaacaggcagcactcatcccctatccctcccaccccaaccctgtcacatacacatacattttctcattctggcactttccctggttctcactgagggtggttgcttctccaaggtgtgtgatttgctctttgtcccccagaatcttttcagccgtgagatgattcatcctgtacatgtgtgcagcagcatttgtcattttttttttttttgccaattcaattaaatctccaccttgggttctgttattgtctatctcctttactagtactttgaacagtagctggtttgtgcctgtagacgtgaggggttgataatgttcataaaacctcagagctagatgcagactcagtgaacgctgggcctagcaaacaccttgatagcccaggctgtaatagaatacctgcacgtaggtctaatagcccagtagttccatttttatgtgcagaagtttaaagaagcttttgtagctcttgcccgccagcacacacacccaccctgccacacctgacctgtagctgtttgagttaggagcaccctttggtctcacttgtgtccccagctgccaatgcaccatctggcatgtggcggtaggtgtgcagtggttgttgtggagtggaagtttaatgtctccatggtgaacctgcctgcctctcacctccctcaggtattcctggtgcagccagcggctggtggaagagcgttactcttggaccagccagctgtcgccggctgacctcatccccctggaggtccctactggtgccagcagccccacccagagagactggcaggagcagttagtggtggggcacaatgtttcctttgaccgagctcatatcagggagcagtacctgatccaggtaaggttcctggggccaactgcaggttctggcatgggatgggccaggagccctaatctcagtggttaggggaggtactcctttcctggcacgtgtctctgttgcctttgctgaagccgcaaggcgcatctgttgaccagctgtgcctctggtctctgtgcctagctgttgtatgtccccgggaaagcctggtataggacctaagttgtcacaaagtaataatggccttcgtctctgtggcattttagagcttagcatgggtcttgaaggttttgagccacagcctgggctcacttcctgccttaaccaccgatgactactgtgagcgccttaacatctctaagtcttagtttccttttttataaaaaggcagacataacagaaatctcataggattaataggagggttggaacaatgcctgcatgtcaaacactcagcactctgcctggtgtatagtagtggcaattcttaattttatgaaaagtgttttttcactggatcttcacaacagccctagaagataggccaggcaggggagagcaaccttaccctatagctgagggtgctgaggctcagacagccttgttgacatgctcagggccacagagcttttgagtggcagggttggggccagaccagatagccctgaaggctttattttggccactctgtatctacgttgctcagagctattgttggaagctgagaaggacttgcacattgggattgagccaggcctgcatcttaaagggtggctaggatttgggaaggcaggccccttacaggtgatggggcaagcatgaacaagcatgaggattctgtatttggtgttgaaggctgtgtgctgggaggggaggctgtttgaggagctgaggtggggctggaggtccacaccaccaagcagtggtgggctggccccacagttgcagcctccctccttcccttcccttttctcctcctcctcctcagggttcccgcatgcgtttcctggacaccatgagcatgcacatggccatctcagggctaagcagcttccagcgcagtctgtggatagcagccaagcagggcaaacacaaggtccagccccccacaaagcaaggccagaagtcccagaggaaagccagaagaggcccagcggtgagagcacactgccggtgggcaggagcatagtgcttgggaccccctctcaccagcccgtctggcccgaggccaggctgatctgccatgtcccttgctctggttccccagatctcatcctgggactggctggacatcagcagtgtcaacagtctggcagaggtgcacagactttatgtaggggggcctcccttagagaaggagcctcgagaactgtttgtgaagggcaccatgaaggacattcgtgagaacttccaggtatggtgctggagggggctctggggacatgggctgtggcacacccctagctgcacttggggagatgcagctgccaggcctgaccctgagagctggtggtggtaatgggatggctgcccaccttgcgccttcctgtcaccttgtgccaggacctgatgcagtactgtgcccaggacgtgtgggccacccatgaggttttccagcagcagctaccgctcttcttggagaggtgagggggagcccatgtgggaatctctgggggtcagtgtgttcctggtacccgggcccactgtaatcaggtggcgctggttctatctcaggttggggaccttagcttttctaggctgaaagaatggagcccttctgttcagtggtgtccatctgggccctggactctggatttgacagaggccctgaaggggagggccatggagttgtgcttgtgtgtcatgtgcacggtcctggtttactgtgcaccttctctaactagatccttagccaagggcttcacatacagcgtggttatgtttattaatgagtctgtcttatgaagtgacccttgtatgctgaaaattcaggtatatttgtaccaaagatatggaaagaaaaaagaagggaggaaaatttgggtgtaacttttgactccctcagagcttaactactaatagcttgctgttggctagaagctttactgataacataatacatattttttatgttatacgtattatatactgtattcttaaagtaagctagataaaagaaaatgtattaagaaaatcatgaggagaaaatatgtttactattcattaggtggaagtggatcatcataaagatatctatccttcacgttgagtaggctgagggcgggggttgggcttgctgtctcgggtggctaaggctgaagaaaataaatgtgtaagtgaacttgcacgatccagacatgtgttgtttaaatgtcagctgtattttaccacccaagttgtgaggttcaggcatgatgtttttcatgtatgggattattagcacagtgcctggcacagagtcattactccacgtgtggcagccattttcacttttgccatctatatttcccacattacccctgaggatgggatgatattgttcccattttatagatgaaagaactgaggctccgagagatggggttgcttacccagggatgagtaacagtagagctgggatttaatgccgtctgacttttgagctgtgccatgtcagtggctgggttgaggcttgctaaaccagctcagggattgggccagtcttgcctcctgtggtcatttatggcagctcctggtgtttgcctccaaggtgtccccacccagtgactctggccggcatgctggagatgggtgtctcctacctgcctgtcaaccagaactgggagcgttacctggcagaggcacagggcacttatgaggagctccagcgggagatgaagaagtcgttgatggatctggccaatgatgcctgccagctgctctcaggagagaggtagccaggccttgggtgggcaggatctaggcaggggactggcaggtgggcggcctagccttcggcttagccttagccctgccctagtggactggctctgtaggtacaaagaagacccctggctctgggacctggagtgggacctgcaagaatttaagcagaagaaagctaagaaggtgaagaaggaaccagccacagccagcaagttgcccatcgagggggctggggcccctggtgatcccatggatcaggaaggtggggagcatgggtgggaggtagggtagggtaggggttgtctctgggaaggtcctgtgattgagggggtccttcgaaaggattgctccagccttctggagatgagcgggtgggagcagatcttattgagagttccttctcctgctcctgattgtcttcccccaccctcacagacctcggcccctgcagtgaggaggaggagtttcaacaagatgtcatggcccgcgcctgcttgcagaagctgaaggggaccacagagctcctgcccaagcggccccagcaccttcctggacaccctgggtgagccctgcccacccccagcagtgtatctagagtctacccttgctccattctcaggacagccctggtctgggttctggcacagaggcatcatgcacatgtatacttattgacctgctgccattcagtcacactgtcttccagtcctattctcatttgctcactctggaccggctcactggactcattcagcacagtgttgtgagcacctgctgtgcaatggcccgtggcagccaccgggtgtacacactggagcatagctcctcctttccagtagttctttttcctaggaggagccaggcacgtagaccagccagtgcagctagtgtccataggtagagttctgactctgcctcgggaaataaatcaagaaggcttccttgagaaggtgccccttcctttgagcctcatagggtggcagagatgagaaaaagggcagccagggtgagcagcagggtgccagctttgcacctgcaagaccctgagagcaagtgtcctgagtgccttgctagtctcaccctgggctcaactctggtgaacagcctgcaagagagcacccagaaggactggtgtttctctagaggggtggggagggcagatctgctccctcctctggtcagttaccctggatgaaatggagcttgggaaggagccctgccctgggtcagggtatgcttttgtgtcctggcttctgactagtccagtgggactgacttagtgtctttgcttttgaaatattcttctagaggattccatgggggtcctggctaaagcatcccagaggaggggatggcggctgtaggctggggtcaccagaaagccccagggctttggagggtgggtggggacattgtgagagagagaaccttccccccaacaactgcccttaccatcgtgacactgctgtcttcctgctgggacgtagatggtaccggaagctctgcccccggctagacgaccctgcatggaccccgggccccagcctcctcagcctgcagatgcgggtcacacctaaactcatggcacttacctgggatggcttccctctgcactactcagagcgtcatggctggggctacttggtgcctgggcggcgggacaacctggccaagctgccgacaggtaccaccctggagtcagctggggtggtctgcccctacaggtaaggcttaggcccaggggaggaaggggctggagcctagggaccccttcccctggctggtcagctcaggctagtggaaagagtttgggttcaagagtctgggttcagaagaagggaaaacaggaaaaaaattaacacacacacacacaccctctctctctctctttctctctctctcactcactcactcactctctctctcactcactcactctctcactcactctctcactcactcactctctcactcactctctcactcactcactctctcactctctcactcactcactcactcactcactctctcactcactctctcactcactcactcactctctctcactctctcactcactcactctctcactcactcactcactcactcactctctcactcactcactcactctctgggttcaggttttttcttccatggctacccttaccctctggatctcagagctctgggagggagtatgttgagatgttcacagtggggaggactaaaggccctactcttgggcccagaagcatagctgccttcacaggaacatgcggagggctgttacaagtagcagggagatgggcttttaaaaaagtgtgtgtatataatttgagtgataattatgggccaagcagtgcttcccttatttgttccccaaggagtcccatgagctagaatggttatccccatgttgtagttgacaaaggcttggttgacttaagatcacagaccctgagctttaggcaggcaggtgttggggagaaacttacagtggcccagaattaagagtcctggctcttcagggcagcctgagtctcttatggggccatgggaccaaaggggataacactggccttgctcctttgagcccgagggtaggtgagcggacaggagccagcctgcagctgggccttgggtcctgtcctcccgctgctgtgctctcagaacttctcttgagacggcagctctgtagtgtaagaggaacttggatttgagtgagacaaggccttgaaccccagcctgctgccagggtgctgtcattttcagtttgtcaatcaatccctgtctaaaacccgggaaagtgctatctggttctgcctcagagctgattctgaggactaaacaaagggaattgtggaaggcactagcaagctgcctggcccagagtgggcatctggtaatcagcggctgctgctgctactgttctctgcccagagccatcgagtccctgtacaggaagcactgtctcgaacaggggaagcagcagctgatgccccaggaggccggcctggcggaggagttcctgctcactgacaatagtgccatatggcaaacggtgagggcaggctctgaacctgagctttggggaggggaggtctctgtattccacccagggaaggggcagcctttgggtgggaggctggcactggtggctcaccccagactggcctgcagtgtctgagtaccatgcagggaggggctggtggattggggcctacccagtcccctgcttcactactttggtccttggactgctccaggtagaagaactggattacttagaagtggaggctgaggccaagatggagaacttgcgagctgcagtgccaggtcaacccctagctctggtgagcagtgcgccggcttgggttctctaggtgggtgctgggtggaaagggcttcctcttgcccacctagttcttcccagccagagttccctaggtcttaagggggttggagatgccaccctgcccctgggaggccccacacgtgttggagcaaggagaaagcctgggtgagacctcatggccatcttgtcatttcccagctgatgacgacagtttcaggcccttttcccaccccctaccccatggcccttgctgaatgcaggtgctggagcagggcctgatataggtgtgtggccctcacagactgcccgtggtggccccaaggacacccagcccagctatcaccatggcaatggaccttacaacgacgtggacatccctggctgctggtttttcaagctgcctcacaaggtgtgtcctgggtcatggcctgtcctgtggtgtttcctcattctgctcaaggcccacagcaggccttcagagtgacacacctgagactttcctttttgtgggaatgactagtagtgggacagagtgtgatttcaggcacatactgtcatctctcagcttttgtttttctaatgaaagtcgggtggcaaggggcatggtggtggaattaaatgacatggggcacgtcgtatgtttggtacgacatctggtacgtgataggtttttccgatttgttattatgcagggagccaggtttgcttgtgtctgtgtgtcttagggggcatgtgtgtgcacgtgtgtgtgtgcgtgcgcgcgtgcgcgcgtgcgtgatacaatcagggatttgcctcagactgctgaggttctgggctcagtgttgggaggagtgcaggtactcacgttggttccccacccaggggtctgccacctgcctccagcccctgcttcctttgctctgtccaggatggtaatagctgtaatgtgggaagcccctttgccaaggacttcctgcccaagatggaggatggcaccctgcaggctggcccaggaggtgccagtgggccccgtgctctggaaatcaacaaaatgatttctttctggaggaacgcccataaacgtatcaggtgggccaccatgggaggagtcctgggatgcctttcccctctcttcccacccagggacccctgactaaccctggattcccacagagggccagcctgactatggtctagaggcctggctacttttggtcctggtgccatggaccttgggcaggtctcccctctagcttcagtttccctgttaatgtaaaaagaatggtgctgtaggaccatgagagcccttcgtagctccaacagaacttcttggtgtaactgctggagccgtgggctatggctgaggaccatggagagctggtggcctgtaagccctgttgggggctgggagctgggtcttctagtctggaatggcaaatgtattcatcttgaaggccatttccaaggtggttgtggccatcagcacactggcgagcagagtgggtgttgggatggtgaagtctgcctgtgtgtaggaagaggcattggtggaaggagcgcctcatggatgccccccggagaggagcggaagctcgctcggaggcctggccggttcccagatggtttatgctcttgattggtgtatcatagggccccagttcttggctgagccagggctcaccttgagtccagttagtgaggctgggtaatggagtatagcagtcctggaggtgggcaggtgagggccatggtgggatgtgggatagattctgcttcccatggctgtgctgagcctcacgttgtctgtccccacagctcccagatggtggtgtggctgcccaggtcagctctgccccgtgctgtgatcaggtatggtctgctgagtggttgtagggataggagaactgaggtgaggtggtaggtcctaaggccaaagcaccctgctaagacccatttccttcccctgcaccccaccaggcaccccgactatgatgaggaaggcctctatggggccatcctgccccaagtggtgactgccggcaccatcactcgccgggctgtggagcccacatggctcaccgccagcaatgcccgggtatgtgacctctgtacctctggcccctgctcttcctctcccaggtctgtagaaactgggctctgagggcctttaggtatttagtgaggatcatgaaaaggaccctgtgatctgggtcaggcaggactctagtcaaatctggcttcatgatttctgtccactccttcagtaaatatgttctgggcacctgctcctggccagaccgtgacaggcgtaatagctacagctctcatggaatttagataggaccgtgtaggtgaggggtctggcatagcgctaggcatagagtagattctttacctgtcacaccaattgctgataggtggccatctctggaactgtggaatttcagcagtgctgtctggcattctctaaagccatcccctcaggaaaggctctagctctttctcagtcaactctggctccaggaatggggtaggaagagtctcatttgggtatctcactcttcccacagcctgaccgagtaggcagtgagttgaaagccatggtgcaggccccacctggctacacccttgtgggtgctgatgtggactcccaagagctgtggattgcagctgtgcttggagacgcccactttgccggcatgcatggtgagcaggagccggggttggggcagcccagcccctcagcatattgacagttctgatgaacattgggcagaatgttcctgagctgcttttctcactcctgcttgtcttccaggctgcacagcctttgggtggatgacactgcagggcaggaagagcaggggcactgatctacacagtaagacagccactactgtgggcatcagccgtgagcatgccaaaatcttcaactacggccgcatctatggtgctgggcagccctttgctgagcgcttactaatgcagtttaaccaccggctcacacagcaggaggcagctgagaaggcccagcagatgtacgctgccaccaagggcctccgctggtgagggtccctctcccatccactttaacacccaggacccgaggcctgctttactgtcctttagtactaccatctgttctatctcctgcccattacttgaactctcacctagcccctctccttccacacctgtgtaacctggttccaggatgatttgtcctattgtgacatttggttgctttatagtcagccttaaacagtttttcctcatgggagtaaagctatacttttggtatactgttaccaagtggtagcatcttgacaattctgattatgctgcataatcaataatacaggggttgcaaactcagatgcctacagggaatgagagcaaatggagtgggtggaagacaggagttgacaggagggcgctgtggcaaactggagcatgtaggctgatgttgatactggagaaagcattaccaggcctccaggttacttagcctagctctccaatttgtttcctctgatcgtactgcatactgtgtgctcagggccttagcagactctctgcagggttccaaaaacattgagggaagagaggtacaacttcctgaggtacagtacactgtccacatttaattagctggctcattgtggaaacttcactttctcgtcaacaactaaaagttaagtatgtgataaatgatatagtggttgatgactataaatgcagggaaggggagctgagtatcgtccagtggataaagtgaggtcgggtaaggctcataccgtgagcagcgtgtgctggtggaggcgagaaaggtggtggggctttagttgtggacacctttgaaagtgtcacaggagtttggactgtgggtgcaggtggtggggaagccatttatgcgagtgacgtgtctctggagccttcaggcgacaagccttgtgaggtctgcaggttagatggaagctgggagttgtctagggttgtggcagttgagaggggtaagccaggcctggctgttgtgttttctgcttcaacaaatgccccctccccttcagggagtagcctattcttacccctatccccccaaatctagagtgatggcccttgctgcctcctgaataaaaggcccgtgttggtcattgggcaattcagtgtctaaagaaacaggacagtaggaatagtggtgcctcctgtgctggagtctttgtcctttattgggctaccatggggtggcccaggctttggggctacaaaagcctgggctgcatctctttctagctccatgatcctaggcaaggcacttagcctctctgagccgtttcttcctctgaataaaagcctttaggggactggcatgatgtcagtgtttttaaaagttgaagtgatatgtgaacattccttgccaaggcactagcgtggcacaggaagcactcccgtggaatgatggtgataacactgcccccaggtatcggctgtcggatgagggcgagtggctggtgagggagttgaacctcccagtggacaggactgagggtggctggatttccctgcaggatctgcgcaaggtccagagagaaactgcaaggaagtaagaaccttctttgtgttaaggatggagggaggggtctgggcttgccccagaagagcttggatgctttgttttttagctttgagatgctgaaagacaaagtctgccctctgtttctggtcccttaggtcacagtggaagaagtgggaggtggttgctgaacgggcatggaaggggggcacagagtcagaaatgttcaataagcttgagagcattgctacgtctgacataccacgtaccccggtgctgggctgctgcatcagccgagccctggagccctcggctgtccaggaagaggtatcttgctacctttggagcatgggcagaggggccccagggagggcagggcagagctccctgtggaccttaccaatgtttgtaggtagggccagagtgaagcttctcttggggcttctaccctggagttaattggtatgtagcatagcccctttcacctctgcccaccttcccttcccagtttatgaccagccgtgtgaattgggtggtacagagctctgctgttgactacttacacctcatgcttgtggccatgaagtggctgtttgaagagtttgccatagatgggcgcttctgcatcagcatccatgacgaggttcgctacctggtgcgggaggaggaccgctaccgcgctgccctggccttgcagatcaccaacctcttgaccaggtatgcggggcccatggcctctagcctggccatgtgctcctatgtggggctttgggtgagcgttccttgggccagactggtcagttttgacttttcatccccctagaagtgaatgtttcagcttatttatttatttctaatttttaaaaagttgtagaagtcctaaaaagactagcctcaattcgtaaaaaaagagttattgggtttgaaaatgtgaaataccaagactgatcattgagggaagcagtgaggttaggggaattgttccgaagggtggtactcacgcttttctatttggaaaatcaaatgacagaagccttttctcatttcatagaaaattgagatgtttgtttttctttctcccataaatgttttctttcttaagtaagtgccaaaagtttgttatttgactgctaacagaaaacactgttaatggggacactcaaatgtgatttttaaaaatatcttatatattttatatattgagttgtattttcttgtagtaaaattcctagttcatatggatgaattaaatattaccgttccatgttgatctgccactcagaaccagtttgggaaccatgatctatcctgattattgggtaaataacagatgtttacaatattcaacattgttcccattgccctcttaatcatcatctccgggaggttatgcttaacaaagctaaaagtcctcatttatgcttcaaactctggcccaattggaagtgatttcgtatattaattaataaagtgtaccaaactgggaaaaaaaaaaaaagtatgttgagtccataattgcatttcagtatctcagtgggaggttaggctgctggatggaaaacagtgctggaccttcacctttcttgacttagctaagtgaacagatggggtgttggtccaggggaagccctgctctaaggggtgtggggtcattgctccaggagtgatgcatctgttcacaggaggggcatgactgtgagagtagattgggtctctttcaggtgcatgtttgcctacaagctgggtctgaatgacttgccccagtcagtcgcctttttcagtgcagtcgatattgaccggtgcctcaggaaggaagtgaccatggattgtaaaaccccttccaacccaactgggatggaaaggagatacgggattccccagggtgagcacaacacatttgttcctcattacacataggatctgaggtggactagaaagtgggtcttggagaacaggaaacttggggccccagagaatccactcttgactcaggctatattctaggctaatttcagtttataaggtgccctgtgtccagagtgaatgtgatatgatgtttcagaaatgaaggcagcagagcttcaaatattctacctgtacctgtcccctacttcaaccacagaagaaatgtttaaagataatttattctatagagtgcattcttgcactctataggtgacagaaaaacaaactgtgctttaaataccaaacaagtaaatcagaaagcttattttctatttaaaatatatctaagacacacttatataaaaagaaaacagaccctcctaacatgtaacattaccgttcgtggcaattgttctcaacctttcactctccttttgaccttagcattaagctcctttgctcacttctgagctctcagttacagttcttgaggtggcatcctaaccaatttgcactatctttcaggtgaagcgctggatatttaccagataattgaactcaccaaaggctccttggaaaaacgaagccagcctggaccatagcactgcctggaggctctgtatttgctcccgtggagcttcatcggggtggtgcaggctcccaaactcaggctttcagctgtgctttttgcaaaagggcttgcctaaggccagccatttttcagtagcaggacctgccaagaagattccttctaactgaaggtgcagttgaattcagtgggttcagaaccaagatgccaacatcggtgtggactacaggacaaggggcattgttgcttgttgggtaaaaatgaagcagaagccccaaagttcacattaactcaggcatttcatttattttttccttttcttcttggctggttctttgttctgtcccccatgctctgatgcagtgccctagaaggggaaagaattaatgctctaacgtgataaacctgctccaaggcagtggaaataaaaagaaggaaaaaaaaga

(19) United States
(12) Patent Application Publication
(10) Pub. No.: US 2007/0277251 A1
(43) Pub. Date: Nov. 29, 2007

(54) Diagnosis and Prediction of Parkinson's Disease

(75) Inventors: Anu Elina Wartiovaara, Helsinki (FI); Petri Tapani Luoma, Veikkola (FI)

Correspondence Address: DECHERT LLP, P.O. BOX 390460, MOUNTAIN VIEW, CA, 94039-0460, US

(73) Assignee: Licentia Oy, Helsinki (FI)

(21) Appl. No.: 10/557,649

(22) PCT Filed: May 19, 2004
(86) PCT No.: PCT/FI04/00307

§ 371 Date: May 1, 2007

(30) Foreign Application Priority Data
May 22, 2003 (FI) 20030778

(51) Int. Cl. A01K 67/027; A01K 67/033; C07H 21/00; C12N 1/00; C12N 15/63; C12N 5/00; C12Q 1/68

(52) U.S. Cl. 800/13; 435/243; 435/320.1; 435/325; 435/6; 536/23.1

(57) ABSTRACT

The present invention provides an in vitro method for diagnosis or prediction of Parkinson's disease in a subject, the method comprising testing for a mutation of a mitochondrial DNA polymerase POLG1 gene of the subject and, where the mutation is detected, diagnosing or predicting Parkinson's disease in the subject. Further, the present invention provides a diagnostic kit for diagnosis or prediction of Parkinson's disease in a subject.

### FIELD OF THE INVENTION

The present invention relates to an in vitro method for diagnosis or prediction of Parkinson's disease in a subject. The present invention further relates to a diagnostic kit for diagnosis or prediction of Parkinson's disease in a subject.

### BACKGROUND OF THE INVENTION

Parkinson's disease [PD, MIM (Mendelian Inheritance in Man) 168600] is the second most common neurodegerative disease with the prevalence of 0.4-2.2% in the Western world. The morphological hallmark of the disease, degeneration of the neurons in substantia nigra, is the end result of various pathogenetical pathways, both genetic and environmental. PD can be divided grossly to two forms, with or without Lewy bodies (LB), which are protein aggregates within the neurons. LBs are associated with PD with dementia. Several triggers are suspected to be linked with PD, such as environmental toxins, oxidative stress and genetic mutations. Mutations causing PD are known in some genes: alpha-synuclein, parkin, DJ-1, ubiquitin C-terminal hydrolase L1 (UCHL1) (Cookson (2003) Neuron, 37:7-10) and NR4A2 (Le et al. (2003) Nat Genet 33:85-89).

The ubiquitin-proteasome pathway is a cellular system that is responsible for degrading damaged or misfolded proteins. Attachment of ubiquitin molecules to lysine residues in a given protein targets it for destruction by a multi-enzyme complex known as the proteasome. Lotharius et al. (2002) Nature reviews, Neuroscience 3:1-11, connected alpha-synuclein, parkin and UHCL1 to the accumulation of dopamine in the cytoplasm leading to the death of nigral dopaminergic neurons in Parkinson's disease. This was attributed to possible defects in the ubiquitin-proteasome pathway. The conclusion of Lotharius et al. (2002) was that all forms of Parkinson's disease might involve dopamine-induced oxidative stress and a disruption of alpha-synuclein folding or processing.

According to Cookson (2003), supra, a shared property of alpha-synuclein mutants is that they form oligomers and protofibrils more readily than wild-type protein. Mutations in parkin result in decreased enzyme activity. DJ-1 is suspected to participate in the oxidative stress. UHCL1 has a ligase activity that promotes addition of ubiquitin to preformed ubiquitin chains on proteins. The hypothesis of Cookson (2003) is that the underlying dysfunction in PD is a failure of udiquitin-mediated protein-degradation. Le at al. (2003), supra, concluded that mutations in NR4A2, encoding member of nuclear receptor superfamily, could cause dopaminergic dysfunction, associated with Parkinson's disease.

The mitochondria are small intracellular organelles, which are responsible for energy production and cellular respiration. They are controlled by two different genomes, the nuclear and the mitochondrial. The mitochondrial genotype is the result of several thousand mitochondrial DNA (mtDNA) copies in each cell. Human mtDNA is a circular double-stranded molecule of 16.6 kb. It encodes 13 subunits of the respiratory chain enzymes, as well as the mitochondrial ribosomal and transfer RNAs. Most of the mitochondrial proteins are, however, encoded by nuclear genes. Consequently a mutation in either genome may result in mitochondrial dysfunction. Mutations of mtDNA have been associated with several human diseases ranging from mild myopathies to severe multisystem disorders. Siciliano et al. (2001), J Neurol Neurosurg Psychiatry 71:685-687, studied the mitochondrial DNA rearrangements in young onset parkisonism with two patients. In addition to PD, one of these patients had liver cirrhosis and in his family, liver cirrhosis, diabetes mellitus and progressive external ophthalmoplegia were diagnosed. The other patient had, in addition to parkinsonism, multiple subcutaneous lipomas. Siciliano et al. (2001) concluded that the pathogenic role of large scale mtDNA deletions in PD is controversial and that mitochondrial impairment could be one of the factors, but not necessarily the primary or the major one, which intervene in the sequence of events involved in the pathogenesis of Parkinson's disease. Cookson (2003), supra, notes that an effect of reduced mitochondrial complex I activity (e.g. by toxins such as rotenone or MPTP) is to decrease cellular ATP levels and may indirectly inhibit the highly ATP-dependent proteasome.

Autosomal dominant progressive external ophthalmoplegia (adPEO) and its recessive form (arPEO) are mitochondrial diseases characterized by two-genome involvement: defective nuclear genes cause accumulation of somatic mtDNA mutations in the patients' postmitotic tissues. Clinical symptoms include PEO and exercise intolerance, and additional symptoms differ in different families: polyneuropathy, hypogonadism, and cataracts have been characterized. Genetic background of ad/arPEO varies in different families, and thus far three distinct nuclear gene defects have been characterized underlying the trait. Mutations in the gene encoding the heart- and muscle-specific adenine nucleotide translocase 1 (ANT1) (Kaukonen et al. (2000) Science 289:782-785) and mutations in Twinkle, a mitochondrial DNA helicase (Spelbrink et al. (2001) Nat Genet 28:223-231), both result in adPEO. Mutations in mtDNA polymerase gamma (POLG) (Van Goethem et al. (2001) Nat Genet 28:211-212) result in adPEO or arPEO. All these proteins can be expected to affect the mtDNA replication, either through altered dNTP metabolism or the replication process as such, and through increasing the error-rate of POLG. The end result is accumulation of multiple mtDNA deletions and point mutations in the patients' tissues, especially in brain, skeletal muscle and the heart.

Previously, Chalmers et al. (1996) Neurol Sci 143:4145, Moslemi et al. (1999) Neurology 53(1):79-84 and Casali et al. (2001) Neurology 56(2):802-805, reported some isolated cases where individual patients had PEO, multiple mtDNA deletions and PD. However, in these studies several patients having PEO, multiple mtDNA deletions, but no PD, were also reported. These patients had various different symptoms, ophthalmoplegia, cataracts, general weakness, and resting tremor among others. Casali et al. (2001) stated that the association of ocular and skeletal myopathy of mitochondrial origin with parkinsonism is apparently unique in the family they had studied. Van Goethem et al. (2003) reported a subgroup of patients with recessive POLG mutations, who manifested the disease as neuropathy. These patients were not reported to have parkinsonism (Neuromuscular Disorders 13:133-142). Thus, results in different studies have been very contradictory. No clear association between mitochondrial DNA mutants and Parkinson's disease has been established and no single mitochondrial genetic defects have been shown to lead to PD.

There is still a strong need to find alternative pathways leading to Parkinson's disease in order to be able to diagnose and predict this disease in more subjects and/or at an earlier stage.

### SUMMARY OF THE INVENTION

It has now surprisingly been found that mutations in the gene encoding the mitochondrial DNA polymerase, and in particular in POLG1 (Genbank Acc no NM_{—}002693) gene (SEQUENCE ID No. 2), are a genetic cause of Parkinson's disease. One or more of these mutations may be involved with the development of Parkinson's disease in a subject. Thus, the presence of these mutations provides a tool for diagnosing or predicting Parkinson's disease.

Accordingly, the present invention provides an in vitro method for diagnosis or prediction of Parkinson's disease in a subject, the method comprising testing for a mutation of a mitochondrial DNA polymerase POLG1 gene (SEQ ID No. 2) of the subject and, where the mutation is detected, diagnosing or predicting Parkinson's disease in the subject.

Further, the present invention also provides a diagnostic kit for diagnosis or prediction of Parkinson's disease in a subject, comprising means for testing for a mutation of the POLG1 gene of the subject and means for determining whether the mutation is indicative for Parkinson's disease.

Further, the present invention provides an isolated polynucleotide comprising at least one mutation of the POLG1 gene, or a fragment of said polynucleotide. A recombinant vector comprising the polynucleotide according to the invention and a cell line and a transgenic non-human mammal or invertebrate comprising the polynucleotide according to the invention are also provided.

### BRIEF DESCRIPTION OF FIGURES

The invention will now be described in further detail, by way of example only, by way of the following detailed description and with reference to the accompanying figures, in which:

FIG. 1 shows PEO-Parkinson pedigrees used in the studies in relation to the invention.

FIG. 2 shows results of PET-analysis of patients studied in relation to the invention and of a control subject.

FIG. 3 shows results of mtDNA analysis of patients studied in relation to the invention.

FIG. 4 shows mutations in POLG1 responsible of Parkinson's disease according to the invention.

### DETAILED DESCRIPTION OF THE INVENTION

The present invention provides an in vitro method for diagnosis or prediction of Parkinson's disease in a subject, the method comprising testing for a mutation of a mitochondrial DNA polymerase POLG1 gene (SEQ ID No. 2) of the subject and, where the mutation is detected, diagnosing or predicting Parkinson's disease in the subject.

Polymerase gamma (POLG) is the mammalian mitochondrial DNA polymerase responsible for replication of the mitochondrial genome. It is, however, only one of tens of proteins involved in mtDNA maintenance, and one of hundreds involved with oxidative phosphorylation. It is therefore surprising that POLG1 pol-domain mutations represent the first gene defect, affecting a mitochondrial protein, which consistently causes parkinsonism. Therefore, oxidative phosphorylation defect alone cannot cause this phenotype. Polymerase gamma is the mitochondrial DNA polymerase, i.e. it replicates mitochondrial DNA. The functional POLG protein consists of two parts, the catalytic subunit, and the processivity factor. These are encoded by two different genes, POLG1 and POLG2, respectively. POLG1 encodes a protein, which can be divided into three parts: polymerase domain (pol), with conserved domains pol a, b and c, which are in charge of DNA synthesis, a spacer region, involved in DNA binding, and an exonuclease domain (exo), with conserved domains exo I, II, and III, which are in charge of proofreading the synthesized strand.

The sequence of a wild-type POLG1 protein (Genbank Acc No. NM_{—}002693) is given as SEQUENCE ID No.1, and the genomic wild-type POLG1 DNA is given as SEQUENCE ID No. 2 (part of genomic clone NT_{—}033276.4, nucleotides 762906-781383, renumbered in SEQ ID No. 2). Sequences are numbered from now on referring to SEQ ID Nos. 1 and 2.

It was noted that dominant POLG1 mutations generally cluster in the region encoding the polymerase (pol) or spacer part of the protein, and recessive mutations affect the region encoding the proofreading part of the protein, the exonuclease (exo) part. In certain embodiments of the present invention, the mutation may modify the function of POLG1 protein (SEQ ID No.1).

Accordingly, a mutation tested in the present invention is preferably located in the pol, spacer or exo part of POLG1 gene, more preferably pol or spacer, including conserved polymerase domains pol a, b and c. In certain embodiments of the invention, the method may comprise testing for a plurality of said gene mutations. In a preferred embodiment, the mutation tested in the present invention is situated between the C-terminus and the exoIII domain of the POLG1 gene, and more preferably in DNA location corresponding to an amino acid location of SEQ ID No. 1 selected from: 43-52, 312, 467, 468, 627, 748, 953, 955, 1105,1143, 1230 and 1236, or in a nucleotide position of SEQ ID No. 2 selected from: 1168-1197, 8258-8259, 10873, 11381, 11818, 15661, 15686, 15790 and 17374.

Mutations in DNA corresponding to the amino acid changes A467T (a mutation of A to T in amino acid position 467 in SEQ ID No. 1), 43-52PolyQ43-50, W312R, N468D, R627Q, W748S, R953C, Y955C, A1105T, E1143G, S1230F and Q1236H, as well as nucleotide changes (in SEQ ID No. 2) 1168-1197 with missing CAGCAG((cag)₈-allele), 8258-8259 with additional G, T10873C, C11381T, T11818C, A15661G, T15686C, G15790A and C17374A can be the cause or associated to PD, alone or two or several together in combination.

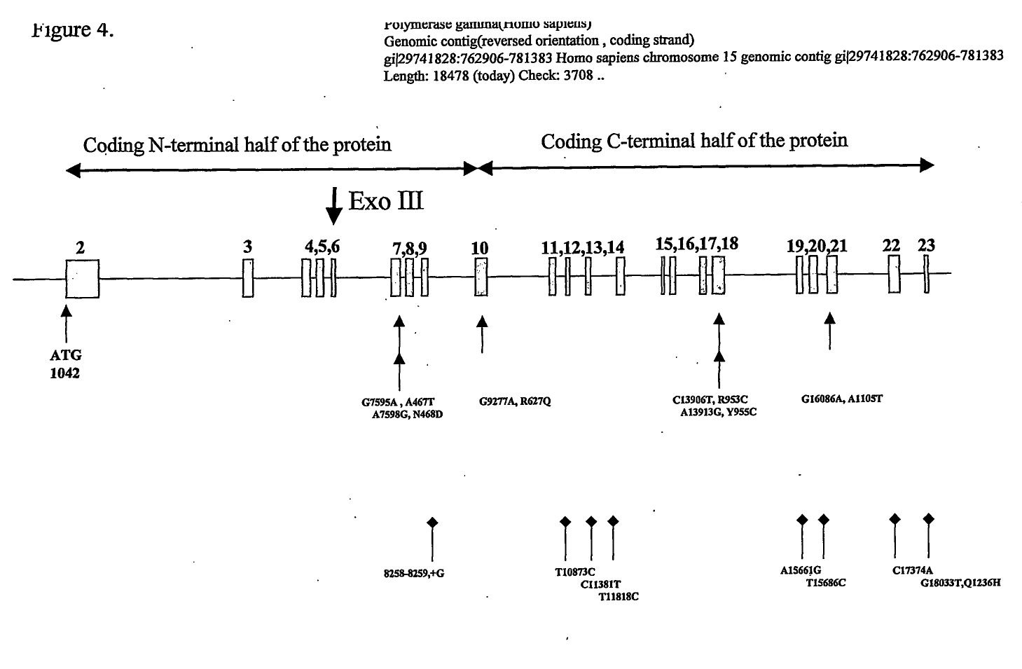

FIG. 4 shows a schematic diagram of POLG1 gene and protein with plurality of mutations according to the invention. According to the present invention the mutations associated with the PD are preferably dominant mutations of pol and spacer domains, more preferably the pol and spacer domains (genomic sequence approx. nt 7554-18478, including the 3′ untranslated region of POLG1 gene of SEQ ID No. 2, and corresponding genomic area with exons and introns), which extends on the protein level carboxyterminally (C-terminally, that is towards the C-terminal half of the protein) from the exo domain to the spacer and pol regions, more preferably from the exoIII domain to the spacer and pol regions, and more preferably carboxyterminally from amino acid 453 of the human POLG1.

The testing for a mutation may be carried out using any suitable method known in the art. Suitable methods comprise, but are not limited to, sequence analysis of the polynucleotide comprising the POLG1 gene (or a copy or transcript thereof) searching for the presence or absence of one or more mutations. This is typically done using an amplification reaction by polymerase chain reaction (PCR) to amplify a target sequence, in this case POLG1 DNA, RNA or cDNA template, or subsequence comprising the mutation utilizing specific primers, or by probe-based detection methods. In the present application a polynucleotide probe or primer includes oligonucleotides and polynucleotides of any sequence length included in the sequence corresponding to the POLG1 genomic gene of SEQ ID No. 2 of less than 18480 bases) or preferably included in the sequence corresponding to the genomic area encoding the protein parts C-terminally from exoIII domain in SEQ ID No. 2 of less than 10925 bases. In a preferred embodiment, the oligonucleotide primers comprise at least 10 contiguous nucleotides, more preferably at least 15 contiguous nucleotides, of a DNA molecule having a sequence recited in SEQ ID Nos. 2-10. Polynucleotides may comprise DNA, RNA, cDNA or a modified DNA or RNA. Suitable primers include short oligonucleotides from exonic and intronic POLG1 sequences, flanking the region of interest. Suitable probes include PCR amplified POLG1-DNA or cDNA fragments or such fragments or complete cDNA or genomic region of POLG1 cloned into bacterial plasmids. Suitable amplification reactions include polymerase chain reaction (PCR). It is further possible to assay protein produced by the POLG1 gene, or a subunit or fragment thereof, for the mutation. This may be achieved by assaying for protein function such as polymerase activity, or by assaying amino acid sequence or composition.

Further, the present invention provides a diagnostic kit for diagnosis or prediction of Parkinson's disease in a subject, comprising means for testing for a mutation of the POLG1 gene of the subject and means for determining whether the mutation is indicative for Parkinson's disease. Means for determining whether the mutation is indicative for PD may include, but are not limited to, photos, tables of figures, written instructions or any other means to be used in evaluation of the result obtained by using the kit.

In one embodiment according to the invention, the diagnostic kit may comprise means for DNA hybridization and/or means for DNA amplification, mutation detection reagents and/or a labeled polynucleotide comprising a nucleotide sequence or one nucleotide complementary to at least part of the gene (cDNA or genomic) encoding POLG1 and containing at least one mutation.

An in vitro assay for a mutation of the POLG1 gene of a subject for diagnosis or prediction of Parkinson's disease in the subject is also provided.

Additionally, the present invention relates to the use of a polynucleotide encoding POLG1 or a fragment of said polynucleotide, preferably comprising at least 10 contiguous nucleotides, more preferably at least 15 contiguous bases, as a probe or primer for diagnosing or predicting Parkinson's disease.

A labeled polynucleotide comprising a nucleotide sequence complementary to at least part of the gene encoding POLG1 and containing at least one mutation is provided. This polynucleotide may be used as a probe or primer in diagnosis or prediction of PD.

Primers and probes, such as RNA, DNA (single-stranded or double-stranded), peptide nucleic acids (PNAs) and their analogs, described herein may be labeled with any detectable reporter or signal moiety including, but not limited to radioisotopes, enzymes, antigens, antibodies, spectrophotometric reagents, chemiluminescent reagents, fluorescent and any other light producing chemicals, and mass labels. Additionally, these probes may be modified without changing the substance of their purpose by terminal addition of nucleotides designed to incorporate restriction sites or other useful sequences, proteins, signal generating ligands such as acridinium esters, and/or paramagnetic particles.

These probes may also be modified by the addition of a capture moiety (including, but not limited to para-magnetic particles, biotin, fluorescein, dioxigenin, antigens, antibodies) or attached to the walls of microtiter trays to assist in the solid phase capture and purification of these probes and any DNA or RNA hybridized to these probes. Fluorescein may be used as a signal moiety as well as a capture moiety, the latter by interacting with an anti-fluorescein antibody.

Any probe or primer can be prepared according to methods well known in the art and described, e.g., in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, probes and primers can be prepared using the Polymerase Chain Reaction (PCR) using primers having an appropriate sequence.

Oligonucleotides may be synthesized by standard methods known in the art, e.g. by use of an automated DNA synthesizer (such as are commercially available from Biosearch (Novato, Calif.); Applied Biosystems (Foster City, Calif.), and so on).

Further, an isolated polynucleotide comprising at least one mutation of the POLG1 gene, or a fragment of said polynucleotide is provided. A recombinant vector comprising the polynucleotide according to the invention and a cell line and a transgenic non-human mammal or invertebrate comprising the polynucleotide according to the invention are also provided. A “mutated gene” or “mutation” or “functional mutation” refers to an allelic form of a gene, which is capable of altering the phenotype of a subject having the mutated gene relative to a subject which does not have the mutated gene. The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

Furthermore, expression and cloning vectors as well as transfected host cells which have a variety of uses are contemplated. First, the cells are useful for producing a mutated POLG1 protein or peptide that can be further purified to produce desired amounts of POLG1 protein or fragments. Thus, host cells containing expression vectors are useful for peptide production. Host cells are also useful for conducting cell-based assays involving the mutated POLG1 protein or POLG1 protein fragments, such as those described below as well as other formats known in the art. Thus, a recombinant host cell expressing a native POLG1 protein is useful for assaying compounds that stimulate or inhibit POLG1 protein function. Host cells are also useful for identifying POLG1 protein mutants in which these functions are affected, as well as for testing functional consequences of POLG1 mutants. Host cells containing the mutations are useful to assay compounds that have a desired effect on the mutant POLG1 protein (for example, stimulating or inhibiting function) which may not be indicated by their effect on the native POLG1 protein.

The expression and cloning vectors usually contain a promoter that is recognized by the host organism and is operably linked to the POLG1 or POLG1 gene nucleic acid. Vector choice is dictated by the organism or cells being used and the desired fate of the vector. Vectors may replicate once in the target cells, or may be “suicide” vectors. In general, vectors comprise signal sequences, origins of replication, marker genes, enhancer elements, promoters, and transcription termination sequences. The choice of these elements depends on the organisms in which the vector will be used and may easily be determined. Some of these elements may be conditional, such as an inducible or conditional promoter that can be turned “on” or “off” when conditions are appropriate. Vectors can be divided into two general classes: Cloning vectors are replicating plasmid or phage with regions that are non-essential for propagation in an appropriate host cell, and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably linked to elements, such as promoters, that signal to the host cell to transcribe the inserted DNA. Some promoters are exceptionally useful, such as inducible promoters that control gene transcription in response to specific factors. Operably linking POLG1 or anti-sense construct to an inducible promoter can control the expression of POLG1 or fragments, or anti-sense constructs. Examples of classic inducible promoters include those that are responsive to a-interferon, heat-shock, heavy metal ions, and steroids such as glucocorticoids (Kaufman R J, Vectors Used for Expression in Mammalian Cells,” Methods in Enzymology, Gene Expression Technology, David V. Goeddel, ed., 1990, 185:487-511) and tetracycline. Other desirable inducible promoters include those that are not endogenous to the cells in which the construct is being introduced, but, however, is responsive in those cells when the induction agent is exogenously supplied.

Promoters are untranslated sequences located upstream (5′) to the start codon of a structural gene (generally within about 100 to 1000 bp) that control the transcription and translation of particular nucleic acid sequence, such as the POLG1 nucleic acid sequence, to which they are operably linked. Such promoters typically fall into two classes, inducible and constitutive. Inducible promoters are promoters that initiate increased levels of transcription from DNA under their control in response to some change in culture conditions, for example the presence or absence of a nutrient or a change in temperature. At this time a large number of promoters recognized by a variety of potential host cells are well known. These promoters are operably linked to POLG1-encoding DNA by removing the promoter from the source DNA by restriction enzyme digestion and inserting the isolated promoter sequence into the vector. Both the native POLG1 promoter sequence and many heterologous promoters may be used to direct amplification and/or expression of the POLG1 DNA. However, heterologous promoters are preferred, as they generally permit greater transcription and higher yields of POLG1 as compared to the native POLG1 promoter. Various promoters exist for use with prokaryotic, eukaryotic, yeast and mammalian host cells, known for skilled artisan.

Expression vectors used in eukaryotic host cells, such as yeast, fungi, insect, plant, animal, human, or nucleated cells from other multicellular organisms, will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding POLG1.

Construction of suitable vectors containing one or more of the above-listed components employs standard ligation techniques. Isolated plasmids or DNA fragments are cleaved, tailored, and religated in the form desired to generate the plasmids required.

Useful in the practice of this invention are expression vectors that provide for the transient or stable expression in mammalian cells of DNA encoding POLG1. In general, transient expression involves the use of an expression vector that is able to replicate efficiently in a host cell, such that the host cell accumulates many copies of the expression vector and, in turn, synthesizes high levels of a desired polypeptide encoded by the expression vector, Sambrook et al., supra, pp. 16.17-16.22. Transient expression systems, comprising a suitable expression vector and a host cell, allow for the convenient positive identification of polypeptides encoded by cloned DNAs, as well as for the rapid screening of such polypeptides for desired biological or physiological properties. Thus, transient expression systems are particularly useful in the invention for purposes of identifying analogs, variants of POLG1 for their sub-cellular localizations that are biologically active. Stable expression means that the gene of interest is incorporated as part of the host cell genome, and therefore the gene is stably expressed. This way clonal cultures can be established, in which long-term effects of POLG1 mutations, as well as treatment focusing to mtDNA replication, can be studied in vitro. In this study, stable cultures are established by viral gene transfer or by direct transfection of DNA constructs, or by any other method known to result in stable expression of the gene of interest.

Propagation of vertebrate cells in culture, typically tissue culture, has become a routine procedure. For more details see, e.g., Tissue Culture, Academic Press, Kruse and Patterson, editors (1973). Examples of useful mammalian host cell lines for propagation are monkey kidney CV1 line transformed by SV40 (COS-7, ATCC CRL 1651); Chinese hamster ovary cells/-DHFR (CHO); human cervical carcinoma cells (HELA, ATCC CCL 2); and canine kidney cells (MDCK, ATCC CCL 34). Viral gene transfer enables the use of mammalian primary cells for transfection. Examples of cell lines useful in connection with the present invention are for example mammalian fibroblasts, myoblasts, myotubes, neural and other stem cells and primary cultured neurons.

Host cells are transfected and preferably transformed with the above-described expression or cloning vectors for POLG1 production and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in section 1.82 of Sambrook et al., supra, or electroporation is generally used for prokaryotes or other cells that contain substantial cell-wall barriers.

Host cells systems may comprise mammalian cells and yeast cells. Other methods for introducing DNA into cells, such as by nuclear microinjection, electroporation, bacterial protoplast fusion with intact cells, or polycations, such as polybrene, polyomithine, and so on, may also be used. For various techniques for transforming mammalian cells, see Keown et al., Methods in Enzymology, 185:527-537 (1990).

Prokaryotic cells used to produce the POLG1 polypeptide related to this invention are cultured in suitable media as described generally in Sambrook et al., supra. In general, principles, protocols, and practical techniques for maximizing the productivity of mammalian cell cultures can be found in Mammalian Cell Biotechnology: a Practical Approach, M. Butler, ed. (IRL Press, 1991).

Gene amplification and/or expression may be measured in a sample directly, for example, by conventional Southern blotting, Northern blotting to quantitate the transcription of mRNA dot blotting (DNA analysis), or in situ hybridization, using an appropriately labeled probe, based on the sequences provided herein. Various labels may be employed, most commonly radioisotopes, particularly ³²P. However, other techniques may also be employed, such as using biotin-modified nucleotides for introduction into a polynucleotide or antibodies recognizing specific duplexes, including DNA duplexes, RNA duplexes, and DNA-RNA hybrid duplexes or DNA-protein duplexes.

Gene expression, alternatively, can be measured by immunological methods, such as immunohistochemical staining of tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like.

Mutated POLG1 nucleic acids are useful for the preparation of POLG1 polypeptide by recombinant techniques exemplified herein which can then be used for production of anti-POLG1 antibodies having various utilities described below.

Antibodies useful for immunohistochemical staining and/or assay of sample fluids may be either monoclonal or polyclonal.

Furthermore, an antibody is provided specifically binding with POLG1, or a fragment thereof. The antibody is useful for the identification for POLG1 in a diagnostic assay for the determination of the levels of POLG1 in a mammal having a disease associated with PD.

Monoclonal antibodies directed against full length or peptide fragments of a POLG1 protein or peptide may be prepared using any well-known monoclonal antibody preparation procedures, such as those described, for example, in Harlow et al. (1988, In: Antibodies, A Laboratory Manual, Cold Spring Harbor, N.Y.). Anti-POLG1 mAbs may be prepared using hybridoma methods comprising at least four steps: (1) immunizing a host, or lymphocytes from a host; (2) harvesting the mAb secreting (or potentially secreting) lymphocytes, (3) fusing the lymphocytes to immortalized cells, and (4) selecting those cells that secrete the desired (anti-POLG1) mAb. The mAbs may be isolated or purified from the culture medium or ascites fluid by conventional Ig purification procedures such as protein A-Sepharose, hydroxylapatite chromatography, gel electrophoresis, dialysis, ammonium sulfate precipitation or affinity chromatography (Harlow et al, supra).

A mouse, rat, guinea pig, hamster, or other appropriate host is immunized to elicit lymphocytes that produce or are capable of producing Abs that will specifically bind to the immunogen. Alternatively, the lymphocytes may be immunized in vitro.

If human cells are desired, peripheral blood lymphocytes are generally used. However, spleen cells or lymphocytes from other mammalian sources are preferred. The immunogen typically includes POLG1 or a POLG1 fusion protein.

Polyclonal Abs can be raised in a mammalian host, for example, by one or more injections of an immunogen and, if desired, an adjuvant. Typically, the immunogen and/or adjuvant are injected in the mammal by multiple subcutaneous or intraperitoneal injections. The immunogen may include POLG1, mutated POLG1, POLG1 fragment or a POLG1 fusion protein.

Examples of adjuvants include Freund's complete and monophosphoryl Lipid A synthetic-trehalose dicorynomycolate (MPL-TDM). To improve the immune response, an immunogen may be conjugated to a protein that is immunogenic in the POLG1 host, such as keyhole limpet hemocyanin (KLH), serum albumin, bovine thyroglobulin, and soybean trypsin inhibitor. Protocols for antibody production are described by (Harlow et al, supra). Alternatively, pAbs may be made in chickens, producing IgY molecules.

A specific POLG1 construct can be used to create knock-in, knock-out and transgenic non-human animals which will serve, for example, as a disease models and facilitates tissue-specific research and design or therapy trials. “Knock-in” animal refers to animal that has had a modified POLG1 gene introduced into its genome and the modified POLG1 gene can be of exogenous or endogenous origin, typically of murine origin.

A “knock-out” transgenic animal refers to an animal in which there is partial or complete suppression of the expression of an endogenous POLG1 gene. This can be for example based on deletion of at least a portion of the gene, replacement of at least a portion of the gene with a second sequence, introduction of stop codons, the mutation of bases encoding critical amino acids, or the removal of an intron junction, and so on. Knock-out animals are made using knock-out constructs, i.e. nucleic acid sequence that can be used to decrease or suppress expression of a protein encoded by endogenous DNA sequences in a cell. In a simple example, the knock-out construct is comprised of a gene, such as the POLG1 gene, with a deletion or disrupting selection cassette in a critical portion of the gene so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo or hygrogene) so that the gene can be represented as follows: POLG1 5′/neol POLG1 3′, where POLG1 5′ and POLG1 3′, refer to genomic or cDNA sequences which are, respectively, upstream and downstream relative to a portion of the POLG1 gene and where neo refers to a neomycin resistance gene. The selection can be any antibiotic, commonly neomycin or hygromycin. In another knock-out construct, a second selectable marker is added in a flanking position so that the gene can be represented as: POLG1/neo/POLG1/TK, where TK is a thymidine kinase gene which can be added to either the POLG1 5′ or the POLG1 3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters. Additional targeting recombination sequences, facilitating in vivo removal of selection cassette (flp/frt recombination) or in vivo excision of a POLG1 sequence, inactivating the gene conditionally in vivo (loxP/cre) are inserted into the targeting construct. In the initial phase, the generated loxP/germ line heterozygous mice are bred further to produce F1 homozygous loxP/loxP mice. These are then bred with transgenic animals, expressing cre-recombinase under a tissue-specific or a constitutive promoter. This promoter can also be activated in vivo, allowing regulation of gene activation on a specific developmental stage. This way, animals with inactivation of POLG1 in a specific tissue, cell type (such as dopaminergic neurons) or whole mouse can be created.

“Transgenic over-expressing mouse” refers to a POLG1 mouse model, which over-expresses a mutant or wild-type POLG1 construct in selected tissues, or constitutively. POLG1 cDNA is cloned into a mammalian expression vector under a specific promoter (for example B-actin), which drives the expression of the protein. This construct is injected into mouse zygote pronuclei, and the founder mice are screened for the presence of the transgene. These mice are then analyzed for the expression of the construct in their various tissues. The founders are bred further to produce F1 mice, which are then bred further to produce homozygous mice for the transgene. The dominant action of the POLG1 gene in question will be monitored in the mouse as a whole, as well as specifically in substantia nigra, associated with Parkinson's disease.

A “non-human animal” is understood to include mammals such as rodents, non-human primates, sheep, dogs, cows, goats, and so on, amphibians, such as members of the Xenopus genus, and transgenic avians, such as chickens, birds, and so on. The term “chimeric animal” is used herein to refer to animals in which the recombinant gene is found, or in which the recombinant gene is expressed in some but not all cells of the animal. The term “tissue-specific chimeric animal” indicates that one of the recombinant POLG1 genes is present and/or expressed or disrupted in some tissues but not in the others. The term “non-human mammal” refers to any member of the class Mammalia, except for humans.

A “transgenic animal” refers to any non-human animal, preferably a non-human mammal, bird or an amphibian, in which one or more of the cells of the animal contain heterologous POLG1 nucleic acid introduced by way of human intervention, such as by transgenic techniques well known in the art. The POLG1 nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the POLG1 polypeptides, for example either wild-type or mutant forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques. The term is intended to include all progeny generations. Thus, the founder animal and all F1, F2, F3, and so on, progeny thereof are included.

Invertebrate models may be created from Drosophila melanogaster and Caenorhabtidis elegans, to study effect of mutations homologous to those mentioned in human POLG1. These models enlighten the role of POLG1 in central nervous system development. The models can easily be stressed by exogenous agents challenging the respiratory chain.

A variety of methods are available for detecting the presence of a particular single nucleotide POLG1 mutation or polymorphic allele in an individual. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms and mutations are summarized below. The method of the present invention is understood to include all available methods.

Nucleic acids can be analyzed by detection methods and protocols relying on mass spectrometry. These methods can be automated. Preferred among the methods of analysis herein are those involving the primer oligo base extension (PROBE) reaction with mass spectrometry for detection (described in the International Applications No. WO 98/20019 and WO 98/20020).

A preferred format for performing the analyses is a chip-based format in which the biopolymer is linked to a solid support, such as a silicon or silicon-coated substrate, preferably in the form of an array. More preferably, when analyses are performed using mass spectrometry, particularly MALDI, nanoliter volumes of sample are loaded on, such that the resulting spot is about, or smaller than, the size of the laser spot. It has been found that when this is achieved, the results from the mass spectrometric analysis are quantitative. The areas under the peaks in the resulting mass spectra are proportional to concentration when normalized and corrected for background. Chips and kits for performing these analyses are commercially available from SEQUENOM under the trademark MassARRAY.™. MassARRAY.™. relies on the fidelity of the enzymatic primer extension reactions combined with the miniaturized array and MALDI-TOF (Matrix-Assisted Laser Desorption Ionization-Time of Flight) mass spectrometry to deliver results rapidly. It accurately distinguishes single base changes in the size of DNA fragments relating to genetic variants without tags.

Multiplex methods allow for the simultaneous detection of more than one polymorphic region in a particular gene or polymorphic regions in several genes. This is a preferred method for carrying out haplotype analysis of allelic variants of the POLG1, or along with allelic variants of one or more other genes associated with PD.

Multiplexing can be achieved by several different methodologies. For example, several mutations can be simultaneously detected on one target sequence by employing corresponding detector (probe) molecules (such as oligonucleotides or oligonucleotide mimetics). The molecular weight differences between the detector oligonucleotides must be large enough so that simultaneous detection (multiplexing) is possible. This can be achieved either by the sequence itself (composition or length) or by the introduction of mass-modifying functionalities into the detector oligonucleotides (see below).

Mass modifying moieties can be attached, for instance, to either the 5′-end of the oligonucleotide, to the nucleobase (or bases), to the phosphate backbone, and to the 2′-position of the nucleoside (nucleosides) and/or to the terminal 3′-position. Examples of mass modifying moieties include, for example, a halogen, an azido, or of the type XR, wherein X is a linking group and R is a mass-modifying functionality. The mass-modifying functionality can thus be used to introduce defined mass increments into the oligonucleotide molecule.

The mass-modifying functionality can be located at different positions within the nucleotide moiety. For example, the mass-modifying moiety, M, can be attached either to the nucleobase, (in case of the C.sup.7-deazanucleosides also to C-7), to the triphosphate group at the alpha phosphate or to the 2′-position of the sugar ring of the nucleoside triphosphate. Modifications introduced at the phosphodiester bond, such as with alpha-thio nucleoside triphosphates, have the advantage that these modifications do not interfere with accurate Watson-Crick base-pairing and additionally allow for the one-step post-synthetic site-specific modification of the complete nucleic acid molecule e.g., via alkylation reactions. Particularly preferred mass-modifying functionalities are boron-modified nucleic acids since they are better incorporated into nucleic acids by polymerases.

Furthermore, the mass-modifying functionality can be added so as to affect chain termination, such as by attaching it to the 3′-position of the sugar ring in the nucleoside triphosphate. For those skilled in the art, it is clear that many combinations can be used in the methods provided herein. In the same way, those skilled in the art will recognize that chain-elongating nucleoside triphosphates can also be mass-modified in a similar fashion with numerous variations and combinations in functionality and attachment positions.

Different mass-modified detector oligonucleotides can be used to detect all possible variants/mutants simultaneously. Alternatively, all four base permutations at the site of a mutation can be detected by designing and positioning a detector oligonucleotide, so that it serves as a primer for a DNA/RNA polymerase with varying combinations of elongating and terminating nucleoside triphosphates. For example, mass modifications can also be incorporated during the amplification process.

A different multiplex detection format is one in which differentiation is accomplished by employing different specific capture sequences which are position-specifically immobilized on a flat surface (such as a chip array). If different target sequences T1-Tn are present, their target capture sites TCS1-TCSn will specifically interact with complementary immobilized capture sequences C1-Cn. Detection is achieved by employing appropriately mass differentiated detector oligonucleotides D1-Dn, which are mass modifying functionalities M1-Mn.

Several methods have been developed to facilitate analysis of single nucleotide polymorphisms and mutations. In one embodiment, the single base polymorphism can be detected by using a specialized exonuclease-resistant nucleotide. In another embodiment of the invention, a solution-based method is used for determining the identity of the nucleotide of a polymorphic site. Alternate embodiments to detect polymorphisms and mutations include Genetic Bit Analysis or GBA.™. as described by Goelet, P. et al. (International Application No. WO92/15712), which uses mixtures of labeled terminators and a primer that is complementary to the sequence 3′ to a polymorphic site, and several primer-guided nucleotide incorporation procedures for assaying polymorphic sites in DNA as described in Nyren, P. et al. (1993) Anal. Biochem. 208:171-175.

For mutations that produce premature termination of protein translation, the protein truncation test (PTT) can be employed as described in van der Luijt, et. al., (1994) Genomics 20:1-4. Briefly for POLG1 PTT, POLG1 RNA is initially isolated from available tissue and reverse-transcribed, and the segment of interest is amplified by PCR, the products are then used as a template for nested PCR amplification with a primer that contains an RNA polymerase promoter and a sequence for initiating eukaryotic translation. Upon SDS gel electrophoresis of translation products, the appearance of truncated polypeptides signals the presence of a mutation that causes premature termination of translation.

Any cell type or tissue may be utilized to obtain nucleic acid samples for use in the diagnostics described herein. In a preferred embodiment, the DNA sample is obtained from a bodily fluid, such as blood, obtained by known techniques (e.g. venipuncture), or saliva. Alternatively, nucleic acid tests can be performed on dry samples (e.g. hair or skin). When using RNA or protein, the cells or tissues that may be utilized must express the POLG1 gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of a POLG1 haplotype and having about 5, 10, 20, 25, or 30 nucleotides around the mutation or polymorphic region. In a preferred embodiment of the invention, several probes capable of hybridizing specifically to other allelic variants involved in a PD are attached to a solid phase support, for example a chip. Mutation detection analysis using chips comprising oligonucleotides, also termed “DNA probe arrays” is described for example in Cronin et al. (1996) Human Mutation 7:244. In one embodiment, a chip comprises all the allelic variants of at least one polymorphic/mutated region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art. They may include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, primer-extension with labeled nucleotides, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of a POLG1 mutant or wild-type haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of a POLG1 PD haplotype is identified by alterations in restriction enzyme cleavage patterns. For example, sample and control DNA is isolated, optionally amplified, digested with one or more restriction endonucleases, and fragment length sizes are determined by gel electrophoresis.

In yet another embodiment, any of a variety of sequencing reactions known in the art can be used to directly sequence the allele. Exemplary sequencing reactions include those based on techniques developed by Maxim and Gilbert ((1977) Proc. Natl. Acad Sci USA 74:560) or Sanger (Sanger et al (1977) Proc. Nat. Acad. Sci USA 74:5463). It is also contemplated that any of a variety of automated sequencing procedures may be utilized when performing the subject assays, including sequencing by mass spectrometry (Cohen et al. (1996) Adv Chromatogr 36:127-162; and Griffin et al. (1993) Appl. Biochem Biotechnol 38:147-159). It will be evident to one of skill in the art that, for certain embodiments, the occurrence of only one, two or three of the nucleic acid bases need be determined in the sequencing reaction. For instance, A-track or the like, for example where only one nucleic acid is detected, can be carried out.

In a further embodiment, protection from cleavage agents (such as a nuclease, hydroxylamine or osmium tetroxide and piperidine) can be used to detect mismatched bases in RNA/RNA or RNA/DNA or DNA/DNA heteroduplexes (Myers, et al. (1985) Science 230:1242). In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at GIT mismatches. According to an exemplary embodiment, a probe based on an allele of a POLG1 locus haplotype is hybridized to a cDNA or other DNA product from a test cell(s). The duplex is treated with a DNA mismatch repair enzyme, and the cleavage products, if any, can be detected from electrophoresis protocols or the like.

In other embodiments, alterations in electrophoretic mobility will be used to identify a POLG1 locus allele. For example, single strand conformation polymorphism (SSCP) may be used to detect differences in electrophoretic mobility between mutant and wild type nucleic acids (Cotton (1993) Mutat Res 285:125-144). Single-stranded DNA fragments of sample and control POLG1 locus alleles are denatured and allowed to renature. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

In yet another embodiment Temperature gradient gel electrophoresis (TGGE) method may be used. TGGE permits the detection of any type of mutation, including deletions, insertions, and substitutions, which is within a desired region of a gene. More details can be found for example in D. Reiner et al. Temperature-gradient gel electrophoresis of nucleic acids: Analysis of conformational transitions, sequence variations and protein-nucleic acid interactions. Electrophoresis 1989; 10: 377-389. However, TGGE does not permit one to determine precisely the type of mutation and its location.

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saild et al. (1986) Nature 324:163; Saiki et al (1989) Proc. Natl. Acad. Sci USA 86:6230).

Alternatively, allele specific amplification technology which depends on selective PCR amplification may be used in conjunction with the instant invention. Oligonucleotides used as primers for specific amplification may carry the mutation or polymorphic region of interest in the center of the molecule so that amplification depends on differential hybridization (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189).

In another embodiment, identification of the allelic variant is carried out using an oligonucleotide ligation assay (OLA), as described in Landegren, U. et al. (1988) Science 241:1077-1080, or OLA based methods as described in Tobe et al. (1996) Nucleic Acids Res 24: 3728. The OLA protocol uses two oligonucleotides which are designed to be capable of hybridizing to abutting sequences of a single strand of a target.

In a modification of OLA, the ligated probe elements act as a template for a pair of complementary probe elements. With continued cycles of denaturation, hybridization, and ligation in the presence of pairs of probe elements, the target sequence is amplified linearly, allowing very small amounts of target sequence to be detected and/or amplified. This approach is referred to as ligase detection reaction. When two complementary pairs of probe elements are utilized, the process is referred to as the ligase chain reaction which achieves exponential amplification of target sequences. (F. Barany, “The Ligase Chain Reaction (LCR) in a PCR World,” PCR Methods and Applications, 1:5-16 (1991)).

The sample, the one or more oligonucleotide probe sets, and a ligase are blended to form a ligase detection reaction mixture. The ligase detection reaction mixture is subjected to one or more ligase detection reaction cycles comprising a denaturation treatment and a hybridization treatment. In the denaturation treatment, any hybridized oligonucleotides are separated from the target nucleotide sequences. The hybridization treatment involves hybridizing the oligonucleotide probe sets at adjacent positions in a base-specific manner to the respective target nucleotide sequences, if present in the sample. The hybridized oligonucleotide probes from each set ligate to one another to form a ligation product sequence containing the target-specific portions connected together. The ligation product sequence for each set is distinguishable from other nucleic acids in the ligase detection reaction mixture. The oligonucleotide probe sets may hybridize to adjacent sequences in the sample other than the respective target nucleotide sequences but do not ligate together due to the presence of one or more mismatches. When hydridized oligonucleotide probes do not ligate, they individually separate during the denaturation treatment.

After the ligase detection reaction mixture is subjected to one or more ligase detection reaction cycles, ligation product sequences are detected. As a result, the presence of the minority target nucleotide sequence in the sample can be identified.

Furthermore, nucleic acid detection kits, such as arrays or microarrays of nucleic acid molecules that are based on the sequence information provided in SEQ ID Nos. 1 and 2 may be provided.

As used herein “Arrays” or “Microarrays” refer to an array of distinct polynucleotides or oligonucleotides synthesized on a substrate, such as paper, nylon or other type of membrane, filter, chip, glass slide, or any other suitable solid support. The microarray can be prepared and used according to the methods described, for example in Lockhart, D. J. et al. (1996; Nat. Biotech. 14: 1675-1680) and Schena, M. et al. (1996; Proc. Natl. Acad. Sci. 93: 10614-10619).

The microarray or detection kit is preferably composed of a large number of unique, single-stranded nucleic acid sequences, usually either synthetic antisense oligonucleotides or fragments of cDNAs, fixed to a solid support. The oligonucleotides are preferably about 6-60 nucleotides in length, more preferably 15-30 nucleotides in length, and most preferably about 20-25 nucleotides in length. For a certain type of microarray or detection kit, it may be preferable to use oligonucleotides that are only 7-20 nucleotides in length. The microarray or detection kit may contain oligonucleotides that cover the known 5′, or 3′, sequence, sequential oligonucleotides which cover the full-length sequence, or unique oligonucleotides selected from particular areas along the length of the sequence. Polynucleotides used in the microarray or detection kit may be oligonucleotides that are specific to a POLG1 gene or genes of interest.

In order to produce oligonucleotides to a POLG1 for a microarray or detection kit, the POLG1 gene(s) is typically examined using a computer algorithm which starts at the 5′ or at the 3′ end of the nucleotide sequence. Typical algorithms will then identify oligomers of defined length that are unique to the gene, have a GC content within a range suitable for hybridization, and lack predicted secondary structure that may interfere with hybridization. In certain situations it may be appropriate to use pairs of oligonucleotides on a microarray or detection kit. The “pairs” will be identical, except for one nucleotide that preferably is located in the center of the sequence. The second oligonucleotide in the pair (mismatched by one) serves as a control. The number of oligonucleotide pairs may range from two to one million. The oligomers are synthesized at designated areas on a substrate using a light-directed chemical process. The substrate may be paper, nylon or other type of membrane, filter, chip, glass slide or any other suitable solid support.

In another embodiment, a POLG1 oligonucleotide may be synthesized on the surface of the substrate by using a chemical coupling procedure and an ink jet application apparatus, as described in the International Application WO95/251 116 (Baldeschweiler et al.). In another embodiment, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more oligonucleotides, or any other number between two and one million which lends itself to the efficient use of commercially available instrumentation.

In order to conduct sample analysis using a POLG1 microarray or detection kit, the RNA or DNA from a biological sample is made into hybridization probes. The mRNA is isolated, and cDNA is produced and used as a template to make antisense RNA (aRNA). The aRNA is amplified in the presence of fluorescent nucleotides, and labeled probes are incubated with the microarray or detection kit so that the probe sequences hybridize to complementary oligonucleotides of the microarray or detection kit. Incubation conditions are adjusted so that hybridization occurs with precise complementary matches or with various degrees of less complementarity. After removal of nonhybridized probes, a scanner is used to determine the levels and patterns of fluorescence. The scanned images are examined to determine degree of complementarity and the relative abundance of each oligonucleotide sequence on the microarray or detection kit. The biological samples may be obtained from any bodily fluids (such as blood, urine, saliva, phlegm, gastric juices, and so on), cultured cells, biopsies, or other tissue preparations. A detection system may be used to measure the absence, presence, and amount of hybridization for all of the distinct sequences simultaneously. This data may be used for large-scale correlation studies on the sequences, expression patterns, mutations, variants, or polymorphisms among samples.

Using such arrays, methods to identify the expression of the POLG1 mutations are provided. In detail, such methods comprise incubating a test sample with one or more nucleic acid molecules and assaying for binding of the nucleic acid molecule with components within the test sample. Such assays will typically involve arrays comprising many genes, at least one of which is a gene used in the present invention and/or alleles of the POLG1 gene used in the present invention. The exemplifying FIG. 4 provides information that has been found in a gene encoding the POLG1 protein used in the present invention. The DNA changes resulting in following amino acid variations were seen, referring to the SEQ ID No. 1: A467T, N468D, R627Q, R953C, Y955C, A1105T and Q1236H. Additional DNA level intronic changes were detected, referring to the SEQ ID No. 2: 8258-8259 +additional G, T10873C, C11381T, T11818C, A15661G, T15686C, C17374A. The changes in the amino acid sequence that these mutations cause can readily be determined using the universal genetic code and the POLG1 protein sequence and are illustrated in attached FIG. 4.

Conditions for incubating a nucleic acid molecule with a test sample vary. Incubation conditions depend on the format employed in the assay, the detection methods employed, and the type and nature of the nucleic acid molecule used in the assay. One skilled in the art will recognize that any one of the commonly available hybridization, amplification or array assay formats can readily be adapted to employ the novel fragments of the Human genome disclosed herein. Examples of such assays can be found in Chard, T, An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of Enzyme Immunoassays: Laboratory Techniques in Biochemistry and Molecular Biology, Elsevier Science Publishers, Amsterdam, The Netherlands (1985).

The test samples used in the present invention include cells, protein or membrane extracts of cells. The test sample used in the above-described method will vary based on the assay format, nature of the detection method and the tissues, cells or extracts used as the sample to be assayed. Methods for preparing nucleic acid extracts or of cells are well known in the art and can be readily be adapted in order to obtain a sample that is compatible with the system utilized.

The person skilled in the art of DNA amplification knows the existence of other rapid amplification procedures such as ligase chain reaction (LCR), transcription-mediated amplification (TMA), self-sustained sequence replication (3SR), nucleic acid sequence-based amplification (NASBA), strand displacement amplification (SDA), branched DNA (bDNA) and cycling probe technology (CPT) (Lee et al., 1997, Nucleic Acid Amplification Technologies: Application to Disease Diagnosis, Eaton Publishing, Boston, Mass.; Persing et al., 1993). The scope of this invention is not limited to the use of amplification by PCR, but rather includes the use of any rapid nucleic acid amplification method or any other procedure which may be used to increase rapidity and sensitivity of the tests. Any oligonucleotide suitable for the amplification of nucleic acids by approaches other than PCR and derived from the POLG1 DNA fragments as well as from selected POLG1 mutation gene sequences included in this document are also under the scope of this invention.

The ligase chain reaction (LCR), sometimes referred to as “Ligase Amplification Reaction” (LAR), described by Barany, Proc. Natl. Acad. Sci., 88:189 (1991) has developed into a well-recognized alternative method for amplifying nucleic acids. In LCR, four oligonucleotides, two adjacent oligonucleotides which uniquely hybridize to one strand of target DNA, and a complementary set of adjacent oligonucleotides, that hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, hybridization and ligation amplify a short segment of DNA. LCR has also been used in combination with PCR to achieve enhanced detection of single-base changes. However, because the four oligonucleotides used in this assay can pair to form two short ligatable fragments, there is the potential for the generation of target-independent background signal. The use of LCR for mutant screening is limited to the examination of specific nucleic acid positions.

The solid-phase minisequencing is based on biotinylation of one PCR-primer. After amplification the product is captured on a streptavidin-coated microtiter well, denatured, and a mutation-specific detection primer is hybridized just adjacent to the nucleotide to be detected. Taq-polymerase extends the primer with a labeled nucleotide (radioactive, typically tritium, fluorescent or any other suitable label), if the nucleotide at the site of mutation matches with the nucleotide provided. The extended primer is denatured and the incorporated radioactivity is measured by scintillation counter or any other detection device for e.g. fluorescent labels. (Syvänen et al. (1990) Genomics 8:684-692).

The self-sustained sequence replication reaction (3SR) (Guatelli et al., Proc. Natl. Acad. Sci., 87:1874-1878 (1990)) is a transcription-based in vitro amplification system that can exponentially amplify RNA sequences at a uniform temperature. The amplified RNA can then be utilized for mutation detection (Fahy et al., PCR Meth. Appl., 1:25-33 (1991)). In this method, an oligonucleotide primer is used to add a phage RNA polymerase promoter to the 5′ end of the sequence of interest. In a cocktail of enzymes and substrates that includes a second primer, reverse transcriptase, ribonuclease H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations and polymorphisms is kinetically limited to screening small segments of DNA (e.g., 200-300 base pairs).

Another embodiment of the invention is directed to kits for detecting a predisposition for developing a PD. This kit may contain one or more oligonucleotides, including 5′ and 3′ oligonucleotides that hybridize 5′ and 3′ to at least one allele of a POLG1 locus haplotype. PCR amplification oligonucleotides should hybridize between 25 and 2500 base pairs apart, preferably between about 100 and about 500 bases apart, in order to produce a PCR product of convenient size for subsequent analysis.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radiolabels, enzymes, fluorescent compounds, streptavidin, avidin, biotin, magnetic moieties, metal-binding moieties, antigen or antibody moieties, and the like.

The kit may, optionally, also include DNA sampling means. DNA sampling means are well known to one of skill in the art and can include, but not be limited to substrates, such as filter papers and the like; DNA purification reagents, lysis buffers, proteinase solutions and the like; PCR reagents, such as 10.times. reaction buffers, thermostable polymerase, dNTPs, and the like; and allele detection means such as the Hinfl restriction enzyme, allele specific oligonucleotides, degenerate oligonucleotide primers for nested PCR from dried blood.

Although particular embodiments have been disclosed herein in detail, this has been done by way of example for purposes of illustration only, and is not intended to be limiting with respect to the scope of the appended claims. The choice of nucleic acid starting material, clone of interest, or library type is believed to be a matter of routine for a person of ordinary skill in the art with knowledge of the embodiments described herein.

### EXAMPLE

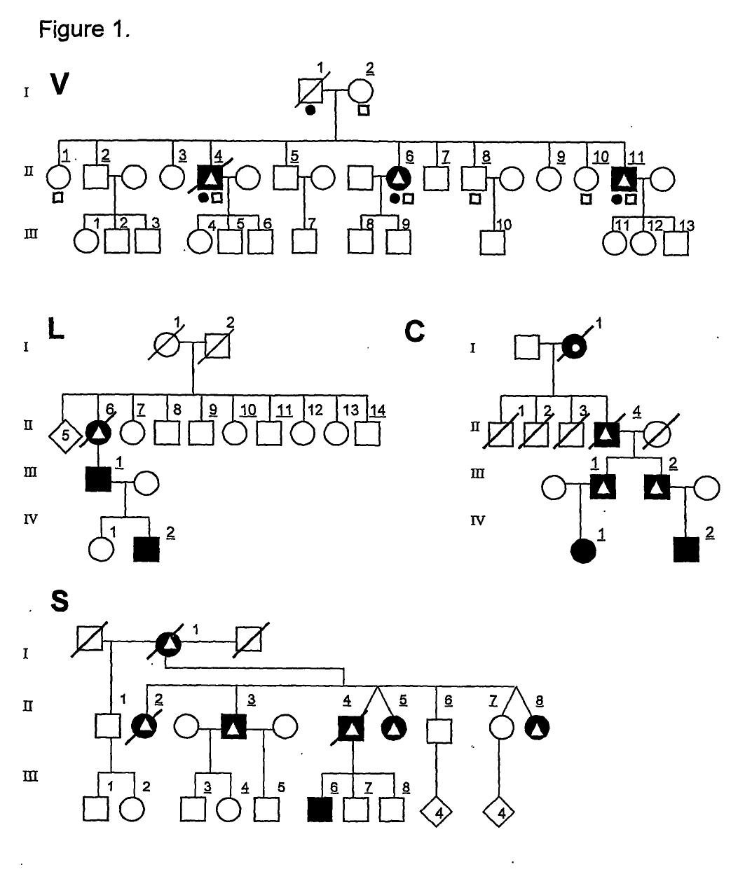

FIG. 1 illustrates the pedigrees used in the studies. In FIG. 1 open symbols are for healthy family members, filled symbols for persons with PEO, filled symbols with white triangle for subjects with PEO and PD, filled symbols with white circle for subjects with PEO, but with no information available for PD. In case of family V, small circle indicates pol mutation in POLG1 and small square indicates exo mutation. Our material consisted of one British (Family C, Chalmers et al. (1996), supra) and one Swedish (Family S, Lundberg (1962) Acta Neurol Scand 38:142-155 and Melberg et al. (1996) Muscle Nerve 19:1561-1569) adPEO family, whose clinical details have been previously described, as well as two Finnish families, one with dominant (family L) and one with recessive pattern (family V) of inheritance. All the studies have been done according to the Helsinki Declaration, and with informed consent. Table 1 summarizes the clinical details of the patients in question, with special emphasis to their PD status.

### Methods

Positron Emission Tomography (PE7)

[¹⁸F] labeled P-CFT (2β-carbomethoxy-3β-(4-fluorophenyl)tropane) (=CFT=WIN 35,428) was synthesized by electrophilic fluorination of 2β-carbomethoxy-3-(4-trimethylstannyl-phenyl)tropane (RBI, Natick, Mass.) as described previously in Rinne et al. (1999) Synapse 31:119-124. The radiochemical purity exceeded 98%. PET scans were performed with the GE Advance PET scanner (General Electric, Milwaukee, Wis., USA) in two-dimensional scanning mode. This device gives 35 transaxial planes, and the transaxial spatial resolution full width half-maximum (FWHM) at a radius of 10 cm in mid plans is about 5 mm axially and transaxially. On average, 150 MBq (4.3 mCi) of ¹β-CFT was injected intravenously. A 60 minute dynamic PET scan was performed 150 minutes after injection. The patients discontinued levodopa at least 12 hours prior to PET scan. The regions of interest (ROIs) were drawn on the head of the caudate, putamen and cerebellum on both hemispheres where the structures were best visualized. The head of the caudate and putamen were located on two consecutive planes. The average of the radioactivity concentrations in these two planes was calculated. Cerebellum was used as a reference, since it contains a negligible amount of dopamine and dopamine receptors. The uptake of [¹⁸F]β-CFT was calculated as the (region-cerebellum)/cerebellum ratio at 180 to 210 minutes. The means of the corresponding left and right hemisphere ROI values were averaged before statistical analyses were performed.

MtDNA Analysis, Respiratory Chain Analyses

Total cellular DNA was extracted from the muscle biopsies and analyzed by Southern Blot exactly as described in Suomalainen et al. (1992) J Clin Invest 90:61-66. As the hybridization probe we used whole human mtDNA, amplified by long polymerase chain reaction (PCR) (Expand-TM Long template, buffer 3).

For biochemical analysis of the respiratory chain enzymes, we used mitochondria, isolated from surgical biopsy specimens of vastus lateralis muscle, within 2 hours, by routine protocols. Oxygen consumption was measured polarographically and the respiratory chain enzyme activities rotenone sensitive NADH:cytochrome c oxidoreductase, antimycin-sensitive succinate:cytochrome c oxidoreductase succinate dehydrogenase, cytochrome c oxidase and citrate synthase were analyzed on isolated mitochondria. As controls we utilized mitochondria isolated from muscle samples from patients (18-73 years) having other than muscle diseases.

Sequence Analysis of the Polymerase Gamma Gene

Total lymphocyte DNA was extracted utilizing standard methods. For sequence analysis, sample from one patient from each family was used to sequence POLG1 gene. The segregation of putative mutations in the families and their presence in control materials (820 anonymous Finnish blood-donors, 1640 control chromosomes) was analyzed by solid-phase mini-sequencing (Syvanen et al. (1990) Genomics 8:684-692).

Intronic PCR primers for POLG1 were as described in Van Goethem et al. (2001), supra. PCR conditions for exons 3-23 of POLG1 were as follows: initial denaturing step at 94° C. for 3 minutes, 30 cycles of 94° C. for 1 minute, 60° C. for 30 seconds, 72° C. for 1 minute and an additional extension step at 72° C. for 5 minutes. Fragments were amplified with DyNAzyme II DNA Polymerase (Finnzymes, Espoo, Finland) in 50 μl of its buffer. Conditions for exon 2 of POLG1 were modified for AmpliTaqGold DNA polymerase (PE Biosystems, Warrington, UK) as follows: preincubation step at 95° C. for 10 minutes, followed by 30 cycles of 95° C. 1 minute, 62° C. 30 seconds, 72° C. for 1 minute, as well as additional extension step at 72° C. for 5 minutes.

PCR fragments were isolated and purified from 1% agarose gels using Qiaex II DNA Extraction Kit (Qiagen, Crawley, UK). Fragments were sequenced by automated nucleotide sequencing using the BigDye terminator Ready Reaction Kit v.3 on a 3100 Genetic Analyzer Automatic Sequencer (Applied Biosystems).

Mutation segregation in families and screening of control samples was performed by solid-phase minisequencing as described in Suomalainen et al. (2000) Mol Biotechnol 15:123-131, using streptavidin-coated microtiter wells (BioBind, ThermoLabsystems). Exonic primers used in minisequencing analysis were:

for exon 18, 5′ATCTACACAGTMGACAGCC3′ (forward, 5′-biotinylated; SEQ ID No. 11), 5′TTAGTAAGCGCTCAGCAAAG3′ (reverse, SEQ ID No. 3), 5′ CAAAGGGCTGCCCAGCACCA3′ (detection, SEQ ID No. 4);

for exon 21, 5′TTTCACCTCTGCCCACCTTC3′ (forward, 5′-biotinylated, SEQ ID No. 5), 5′ CATGGCCACMGCATGAGGT3′ (reverse, SEQ ID No. 6), 5′GAGGTGTMGTAGTCMCAG3′ (detection, SEQ ID No. 7);

for exon 7, 5′ACCAGMCTGGGAGCGTTAC3′ (forward, 5′-biotinylated, SEQ ID No. 8), 5′ CTACCTCTCTCCTGAGAGCA3′ (reverse, SEQ ID No. 9), 5′GAGCAGCTGGCAGGCATCAT3′ (detection, SEQ ID No. 10).

The PCR conditions were as follows: preincubation step at 94° C. for 3 minutes, followed by 30 cycles of 95° C. for 30 seconds, annealing at 48° C., 61° C. or 50° C. (exons 18, 21 and 7, respectively) for 30 seconds, 72° C. for 30 seconds, as well as additional extension step at 72° C. for 5 minutes.

### Results

Clinical Examination

Patients

In all pedigrees, the patients invariably had PEO, exercise intolerance, cataracts and sensory polyneuropathy. All the eldest patients studied had parkinsonian symptoms. The age of onset of the parkinsonian symptoms varied between different families, from early onset 36-46 years in families V and L to predominantly late-onset 58-72 years in families S and C. The age of onset for PEO symptoms varied greatly, from 10 to 49 years of age, but most commonly at the age of 20-30 years. The early-onset PEO did not correlate with early-onset PD. The polyneuropathy was mainly sensory, sometimes with a motor component, and tendon reflexes were always absent. The cataracts were usually noted in neuro-ophthalmologic examination, and in a 36 years old patient they were of congenital type. Secondary amenorrhea was cosegregating with the disease in family S; in family C the patients were male, and in family L, the sole female patient had had her only child young, and no records were available concerning her menstruation. In family V, the patient 11/6 had an early menopause. All patients, who were tested, had RRFs and cytochrome-c-negative fibers in the muscle sample. The quantity varied between 3-6% of total fibers.

Family Members with no PEO

The medical histories of the subjects in the pedigree L included ventricular ulcer, cholecystitis, spondylarthritis, acute thyreoiditis, and asthma, epidermolytic skin disorder, and ischemic heart disease. In the neurological examination all these subjects were healthy, with no signs of exercise intolerance. Two aged subjects had few fibres of reduced cytochrome c oxidase-reactivity in histochemical assay, but no RRFs.

PET-Analysis

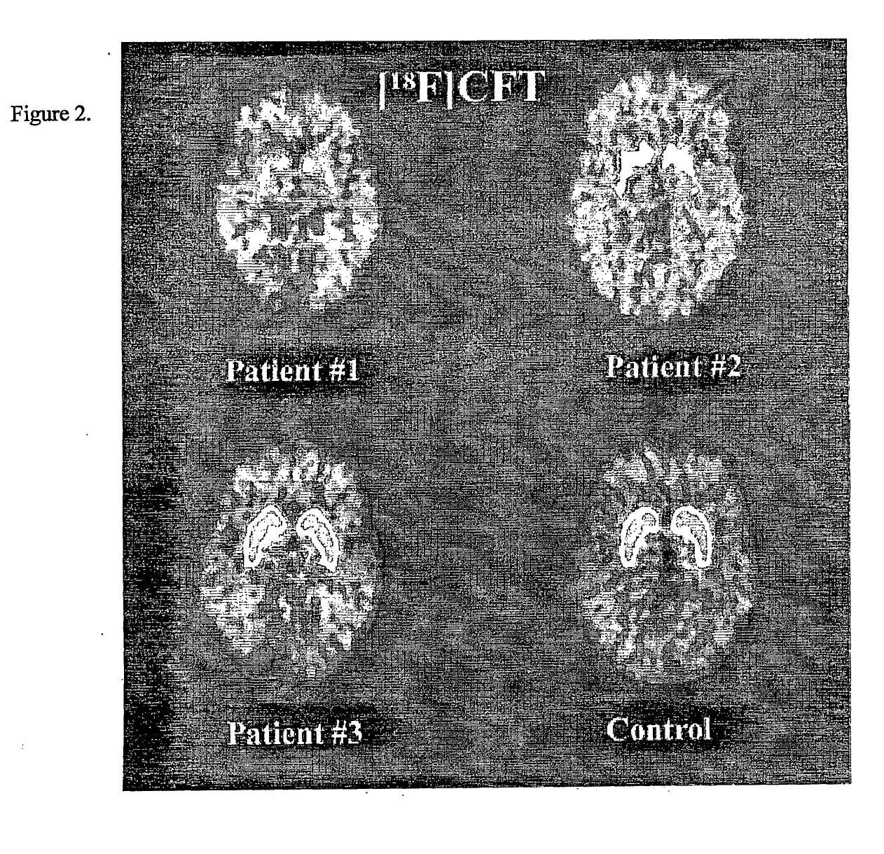

FIG. 2 shows results of the PET-analysis. Patients II-6 and II-11, as well as their healthy brother were studied with PET. Reduced putaminal and caudate [¹⁸F]β-CFT uptake was seen in two patients with multiple mitochondrial DNA deletions (#1 and #2, Patient V/II-6 and V/II-11, respectively), and normal uptake in their brother (Subject #3) with wild-type mitochondrial genome. A PET scan of one non-related control subject (Control) is shown as a reference. The uptake values of the patients were below −3SD from the mean of the controls both in the putamen and caudate nucleus. [¹⁸F]β-CFT uptake values of the healthy brother were similar to those in the controls (Table 2).

MtDNA Analysis, Respiratory Chain Enzyme Analysis

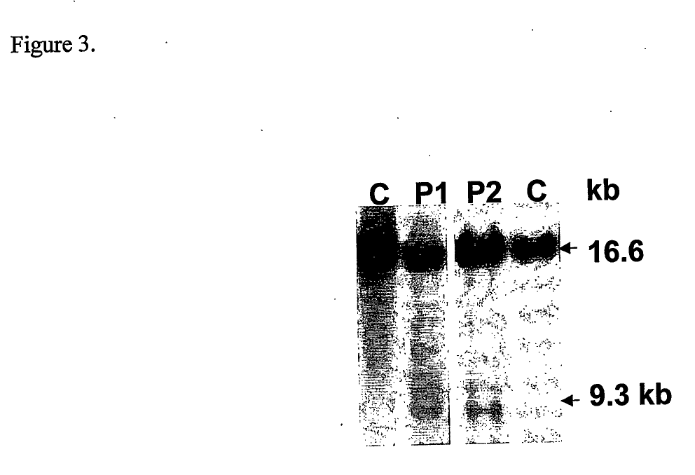

FIG. 3 shows results of mtDNA analysis. Southern blot analysis of total muscle DNA, hybridized with radioactively labelled total human mtDNA, revealed normal mtDNA of 16.6 kb in all samples, but in patients' samples (P1 and P2) also multiple additional fragments of smaller molecular weight, representing mtDNA molecules harbouring different-sized deletions. FIG. 3 shows the finding in Finnish families, and those for British and Swedish families have been published previously (Suomalainen et al. (1992), supra). Southern blot analysis revealed the presence of multiple mtDNA deletions in the samples of all the affected subjects. None of the healthy subjects had mtDNA deletions.

Table 3 summarizes the biochemical analyses of the respiratory chain enzyme activities of three affected subjects from the family V. The activities were mostly within the low normal or normal range. Oxygen consumption utilizing Complex I-dependent substrates was slightly reduced within patient V/II-11, but otherwise normal in all samples. The activities of the respiratory chain enzymes were normal in those clinically unaffected family members, who were analyzed.

Sequence Analysis of the Polymerase Gamma Gene

The entire POLG1 gene was sequenced in two families, and solid-phase minisequencing detection for the specific mutation Y955C (Van Goethem (2001), supra, and Lamantea et al. (2002) Ann Neurol 52:211-219) was used in two families.

Mutations found in this study are shown in FIG. 4. Families C, S and L were shown to carry A->G change at position 2864 (numbering starting from first ATG of SEQ ID No. 1, Genbank Acc no NM_{—}002693), resulting in a replacement of a highly conserved tyrosine to a cysteine at the amino acid position 955. FIG. 1 shows the mutation carriers and those who were analyzed. In family L, Subject II/6 was considered to be a founder, since none of her siblings were affected or were carriers of the mutation.

Family V showed inheritance best compatible with autosomal recessive transmission, and two new mutations were identified to cosegregate with the disease manifestation. The first mutation was a A->G change at the nt 7598, and predicts an amino acid change from asparagine to aspartic acid at position 468, which is located in the exonuclease domain of the enzyme. The second mutation was a nt 16086 G->A change, predicting change of alanine to threonine at position 1105, located in the polymerase part near pol c motif. Only affected subjects of this family were compound heterozygous whereas healthy family members carried either wild type alleles or one of the mutated alleles (FIG. 1). Neither of these mutations was found in screening of 820 Finnish healthy controls.

Cell Transfection Experiment with Mutated POLG1 Construct

POLG1 cDNA is cloned into retroviral expression constructs (pLXSN, pLXSH, PBABE, pBMN-IRES GFP, LentiLox), and these are transfected by lipofectamine transfection to specific retrovirus packaging lines (GPE-86, PA317, Phoenix eco, ampho, 293T) to produce retroviruses carrying POLG1 wild-type or mutants. This viral supernatant is used to transduce any dividing primary mammalian cell lines, including myoblasts, fibroblasts, lymphoblasts, and stem cells (neural stem cells, that can be differentiated to dopaminergic neurons after transduction; dopaminergic neurons are affected in PD). Stable expressing lines are selected based either on antibiotic resistance or GFP fluorescence. Replication ability of mutant POLG1 are assayed, as well as effects on cell viability, growth, signaling and differentiation.

Transient transfection of easily transfectable cell lines, such as BHK and COS cells, are performed by lipofectamine tranfection of the expression plasmids into the cells. Transient transfection is scored by fluorescence of tagged proteins, or by specific POLG1 antibodies.

Generation of POLG1 Mutated Transgenic Mice

The optimal model of studying dominant effect of POLG1 Y955C is either by generation of overexpressing transgenic mice, or by replacing wild-type allele of the mouse by knock-in construct into the mouse POLG1 locus. Transgenic mice can be made by utilizing a mammalian expression vector, expressing wild-type POLG1 or the mutant Y955C form under a cell-type-specific (for example tyrosine hydroxylase promoter, dopaminergic-neuron specific) or a constitutive promoter (such as beta-actin). This construct is purified and injected into the pronucleus of mouse zygotes, and injected into pseudopregnant female mice. The offspring are screened for transgene presence and copy number from tail biopsies. F1 offspring are mated further to produce F2 offspring, to increase the expression level of transgene.

Knock-in mice may be made by making a targeting construct as follows: Mouse POLG1 genomic gene is cloned from mouse lambda genomic library. Restriction fragments of the POLG1 locus are cloned into a plasmid or lamda phage vector, such as DelBoy or lambda 2TK. Neo marker gene is cloned into a large intron to facilitate selection of targeted clones in G418 antibiotic. Neo is flanked by two frt-recognition sites, to allow removal of neo-gene after successful targeting. Two loxP sites are inserted around exon/s, allowing in vivo inactivation of the knock-in allele (knock-out). HSV-TK gene is cloned 3′ from right targeting arm, to allow negative selection for incomplete homologous recombinants. Patient POLG1 mutation, for example Y955C, is introduced into the corresponding site of the mouse gene by site-directed mutagenesis. The correct cloning of targeting construct is controlled by DNA sequence analysis.

The purified construct is transfected by electroporation into mouse embryonal stem cells, and correctly targeted clones are identified by their ability to grow in G418 (neo) and ganciclovir (TK) selection antibiotics. Correct targeting of the construct into POLG1 locus is confirmed by DNA extraction, PCR and Southern Blot analyses. Positive clones are transiently transfected with flp-recombinase expression plasmid, which removes neo-cassette. Alternatively, the neo gene can be later removed by crossing targeted mice with flp-expressing transgenic mouse line. The removal of neo gene is confirmed by DNA extraction, PCR and Southern Blot analyses. The ES-clones are processed to aggregation chimeras and these are injected into pseudopregnant female mice. The positive offspring are analyzed from tail biopsies, and germ line transmission confirmed by further breeding. Heterozygous POLG1-mutant mice are bred further to produce homozygous POLG1 mutant mice. The phenotype of the produced mice is followed by biochemical tests on mitochondrial function, by molecular genetic analysis of mitochondrial DNA, by functional analyses of primary cell lines created from the mice, by morphological characterization of histological features and by functional and neuroimaging means of mice in vivo. Knock-out inactivation of POLG1 can be achieved by crossing the homozygous POLG1 loxP/loxP mice with cre-recombinase expressing transgenic mice. Depending on where cre is expressed, either tissue-specific knock-outs or whole-mouse inactivation of POLG1 is achieved. The mouse model provides an in vivo model for development of treatment for PD caused by POLG1 and related proteins.

TABLE 1
Clinical symptoms and signs of the patients.

| | | | | | | | | | | Flexor | |
|---|---|---|---|---|---|---|---|---|---|---|---|
| | Age of onset | | Resting | | Levodopa | | | Muscle | Additional | plantar | |
| Family C | PEO | PD | tremor | Rigidity | response | Ptosis | PEO | weakness | symptoms | reflexes | Neuropathy |
| I-1 | na | na | + | na | na | + | na | na | na | na | na |
| | | | upper | | | | | | | | |
| | | | limbs | | | | | | | | |
| II-4 | na | <50 | + | + | na | + | + | + | na | na | + |
| | (died 69) | | | | | | | | | | |
| III-1 | 30 | 68 | + | − | + | + | + | Face, neck, | cataracts | absent | + |
| | | | upper | | | | | limbs | | | lower limbs: |
| | | | limbs | | | | | | | | vibr, JPS |
| | | | | | | | | | | | impaired |
| III-2 | 49 | 72 | + | + | + | + | + | Progressive | cataracts | Absent | + |
| | | | upper | | | | | in legs, | | | lower limbs: |
| | | | limbs | | | | | face, neck, | | | vibr, JPS |
| | | | | | | | | fatigue | | | impaired |
| | | | | | | | | upper limbs | | | |
| IV-1 | Na | na | na | na | Not given | + | + | na | na | na | na |
| IV-2 | 28 | − | − | − | − | + | + | Exercise | Pigmentary | Absent | + |
| | | | | | | | | intolerance | retinopathy | | lower limbs: |
| | | | | | | | | | | | vibr, JPS |
| | | | | | | | | | | | impaired |

| | | | | Rigidity, | | | | | | | |
|---|---|---|---|---|---|---|---|---|---|---|---|
| | Age of onset | | Resting | hypo- | Levodopa | | | Muscle | Additional | Achilles | |
| | PEO | PD | tremor | kinesia | response | Ptosis | PEO | weakness | symptoms | reflex | Neuropathy |
| Family S | | | | | | | | | | | |
| I-1 | 30 | 7^{th} | − | + | + | + | + | + | Menopause | present | none |
| | | decade | | | | | | | age 44, | | |
| | | | | | | | | | hypoacusia | | |
| II-2 | 27 | <59 | + | + | + | + | + | + limbs | Hypogonadism | absent | + lower |
| | | | | | | | | | cataracts, | | legs, vibr |
| | | | | | | | | | presbyacusis, | | impaired |
| | | | | | | | | | ataxia | | |
| II-3 | 35 | 6^{th} | − | + | + | + | + | + | Cataracts, | absent | + feet |
| | | decade | | | | | | | ataxia | | |
| II-4 | 25 | 58 | + | + | Not | + | + | + | Cataracts, | absent | + feet |
| | | | | | given | | | | ataxia | | |
| II-5 | 23 | 59 | + | + | + | + | + | + | Mental | absent | + feet |
| | | | neck, arms | | | | | | retardation, | | |
| | | | | | | | | | cataracts, | | |
| | | | | | | | | | ataxia, | | |
| | | | | | | | | | primary | | |
| | | | | | | | | | amenorrhea | | |
| II-8 | 21 | 6^{th} | + | −(hypo- | + | + | + | − | Hypogonadism | absent | none |
| | | decade | neck | mimia+) | | | | | Cataracts, | | |
| | | | | | | | | | ataxia | | |
| Family V | | | | | | | | | | | |
| II-4 | 30 | 36 | + | + | nd | + | + | + limbs, | cataracts | absent | + sensory |
| | (died 51) | | | | | | | pharynx, | | | axonal; |
| | | | | | | | | exercise | | | upper limbs, |
| | | | | | | | | intolerance | | | moderate motor |
| II-6 | <33 | 46 | + | + | moderate | + | + | + face, | Periodic | absent | none |
| | | | upper limbs | | | | | pharynx | depression, | | |
| | | | | | | | | general | menopause | | |
| | | | | | | | | fatigue | before 40 | | |
| II-11 | 21 | 36 | Periodic | − | + | + | + | Exercise | | absent | + sensory |
| | | | +, upper | | | | | intolerance, | | | axonal |
| | | | limbs | | | | | muscle pain | | | |
| Family L | | | | | | | | | | | |
| II-6 | 10 | 46 | Right leg, | + | + | + | + | + | Psychiatric | absent | none |
| | (died 65) | | right arm | | | | | | symptoms, | | |
| | | | | | | | | | dementia, | | |
| | | | | | | | | | cataracts | | |
| III-1 | 33 | − | − | − | Not | + | − | − | − | Weak | none |
| | | | | | given | | | | | reflex | |

Abbreviations: na, not analyzed, or data not available. JPS, joint position sense; vibr, vibration sense.

TABLE 2
[¹⁸F]β-CFT uptake values in patients and controls. The
results are expressed as (region-cerebellum)/cerebellum ratio.

| | UPDRS score | Putamen Ratio | Caudatus Ratio |
|---|---|---|---|
| | (motor part) | (% of normal) | (% of normal) |
| Patient V/II-6 | 29 | 1.3* (32) | 1.0* (27) |
| Patient V/II-11 | 19 | 1.1* (28) | 0.9* (24) |
| Subject V/II-7 | 0 | 4.1 (102) | 3.7 (100) |
| Controls (mean; SD) | 0 | 4.0; 0.6 (100) | 3.7; 0.5 (100) |

*Values below the 3 SD limit from the mean of the controls. Controls include 14 healthy volunteers (6 women, 8 men, mean age 56 years). They were scanned with the same scanner as the study subjects and the scans of the controls were analysed by the same investigator as those of the patients. UPDRS score (United Parkinson's Diseased Rating Scale) was determined on the day of PET scanning.

TABLE 3
Oxygen consumption and biochemical activities of the
respiratory chain enzymes in isolated muscle mitochondria

| | | | |
|---|---|---|---|
| Patient | Patient | Patient | |
| V/II-2 | V/II-6 | V/II-11 | Controls |

Oxygen consumption nmol/O/min mg of mitochondrial protein

| | | | | |
|---|---|---|---|---|
| Respiratory control rate | 3.1 | 7.0 | 2.1 | 5.1 ± 2.0 |
| Pyruvate + malate | 81 | 98 | 57 | 103 ± 37 |
| Succinate + rotenone | 114 | 124 | 109 | 80 ± 22 |
| Ascorbate + TMPD | 735 | 663 | 786 | 402 ± 141 |
| Palmitoyl CoA + malate | 68 | 71 | 30 | 51 ± 15 |

Respiratory chain enzyme activities nmol/min/mg protein

| | Patient V/II-2 | | Patient V/II-6 | | Patient V/II-11 | | Controls | |
|---|---|---|---|---|---|---|---|---|
| | | Ratio to CS | | ratio to CS | | ratio to CS | | ratio to CS |
| CI + IIIₐ | 187 | 0.13 | 236 | 0.16 | 291 | 0.20 | 298 ± 79 | 0.31 |
| CII + IIIₐ | 553 | 0.39 | 260 | 0.17 | 950 | 0.65 | 363 ± 63 | 0.38 |
| CII_{c} | 173 | 0.12 | 249 | 0.17 | 215 | 0.15 | 228 ± 66 | 0.24 |
| CIV_{b} | 1518 | 1.06 | 2531 | 1.7 | 5157 | 3.5 | 2813 ± 639 | 2.95 |
| Citrate synthase_{d} | 1426 | | 1474 | | 1458 | | 953 ± 192 | |

Values correspond to reductionₐ or oxidationₐ of cytochrome c, reduction of dichlorophenol indophenol_{b} and formation of citrate_{d}, expressed as nmol/min mg mitochondrial protein. Control values are mean ± SD.ₑ ratio to CS: the individual complex activity values were calculated compared to the mitochondrial matrix enzyme, citrate synthase. The ratio for controls was calculated from the controls' mean.

```
SEQUENCE ID No. 1
POLG1 protein sequence
      1 MSRLLWRKVA GATVGPGPVP APGRWVSSSV PASDPSDGQR RRQQQQQQQQ
    51 QQQQQPQQPQ VLSSEGGQLR HNPLDIQMLS RGLHEQIFGQ GGEMPGEAAV
  101 RRSVEHLQKH GLWGQPAVPL PDVELRLPPL YGDNLDQHFR LLAQKQSLPY
  151 LEAANLLLQA QLPPKPPAWA WAEGWTRYGP EGEAVPVAIP EERALVFDVE
  201 VCLAEGTCPT LAVAISPSAW YSWCSQRLVE ERYSWTSQLS PADLIPLEVP
  251 TGASSPTQRD WQEQLVVGHN VSFDRAHIRE QYLIQGSRMR FLDTMSMHMA
  301 ISGLSSFQRS LWIAAKQGKH KVQPPTKQGQ KSQRKARRGP AISSWDWLDI
  351 SSVNSLAEVH RLYVGGPPLE KEPRELFVKG TMKDIRENFQ DLMQYCAQDV
  401 WATHEVFQQQ LPLFLERCPH PVTLAGMLEM GVSYLPVNQN WERYLAEAQG
  451 TYEELQREMK KSLMDLANDA CQLLSGERYK EDPWLWDLEW DLQEFKQKKA
  501 KKVKKEPATA SKLPIEGAGA PGDPMDQEDL GPCSEEEEFQ QDVMARACLQ
  551 KLKGTTELLF KRPQHLPGHP GWYRKLCPRL DDPAWTPGPS LLSLQMRVTP
  601 KLMALTWDGF PLHYSERHGW GYLVPGRRDN LAKLPTGTTL ESAGVVCPYR
  651 AIESLYRKHC LEQGKQQLMP QEAGLAEEFL LTDNSAIWQT VEELDYLEVE
  701 AEAKMENLRA AVPGQPLALT ARGGPKDTQP SYHHGNGPYN DVDIPGCWFF
  751 KLPHKDGNSC NVGSPFAKDF LPKMEDGTLQ AGPGGASGPR ALEINKNISF
  801 WRNAHKRISS QMVVWLPRSA LPRAVIRHPD YDEEGLYGAI LPQVVTAGTI
  851 TRRAVEPTWL TASNARPDRV GSELKAMVQA PPGYTLVGAD VDSQELWIAA
  901 VLGDAHFAGM HGCTAFGWMT LQGRKSRGTD LHSKTATTVG ISREHAKIFN
  951 YGRIYGAGQP FAERLLMQFN HRLTQQEAAE KAQQMYAATK GLRWYRLSDE
1001 GEWLVRELNL PVDRTEGGWI SLQDLRKVQR ETARKSQWKK WEVVAERAWK
1051 GGTESEMFNK LESIATSDIP RTPVLGCCIS RALEPSAVQE EFMTSRVNWV
1101 VQSSAVDYLH LMLVAMKWLF EEFAIDGRFC ISIHDEVRYL VREEDRYRAA
1151 LALQITNLLT RCMFAYKLGL NDLPQSVAFF SAVDIDRCLR KEVTMDCKTP
1201 SNPTGMERRY GIPQGEALDI YQIIELTKGS LEKRSQPGP
```

```
SEQUENCE ID No. 2
POLG1 genomic gene locus
According to gi|29741828:762906-781383 Homo sapiens chromosome
15 genomic contig gi|29741828:762906-781383 Length: 18478
        1 GCGGACCGGC CGGGTGGAGG CCACACGCTA CCCCGAGGCT GCGTAGGCCG
      51 CGCGAAGGGG GACGCCGTGC CGTGGGCCTG GGGTCGGGGG AGCAGCAGAC
    101 CGGGAAGCAC CGTGAGGACC GAGGTTTCCG GCGGGGTCGG CGGCGGGGAG
    151 GCCGGGTCGC TGAGCGACGG CGCGGCCCCT CCCTCTCCAG TCAGGGAGCG
    201 AGGCCCGGAG CAGGGCGGCG GCTAGTCCCA GGGCGCACCG CGGCGCCTCT
    251 GCCGGGCGCA GGCGGGCGGC GGGGCGCACG GGGGTGGCCG CCGACTCCTC
    301 CTGCAGGACG CTCTCGGCCG GGTGGGCCGT GGTCCGGGTG TGGGTGTGGG
    351 TCCCGGGGGA CGGCGGCCCA CCCTGCGGGT TCGAATCCGG GCGCTGGCAC
    401 CTCTCGACGC TAGGCCCGCG CCGGTCGCGG TAATGGCAGC CACCATTTGC
    451 CGAGCGCTTG CCAAGAGCAG GGCCGCACGA CATAGGCGCC CTGTGTCCCC
    501 CAGACAGCAG CCCGGTGTGA CAGGCAGAAT CCGTAATCCC ATTTTACAGA
    551 ATAGGATATC AGGGGCTAAG GAGCTTTGCC CAAGGTCACA CAGCTCGAGA
    601 GAGCCAGAAG GGGGGTTCAA AACCGCGTCG CCCTACTCCA GATACTGCTC
    651 TCTTACTCGC TGCCCTCGGC TTCCCCACGT GGGTTCACTG ACGAAGTTGC
    701 GTGGACCCCG GTTCCCCCAG GAGGGGTATT GACGTTTCCC AAGTTTTGAG
    751 GCTTAACGGA AAATGCAACT GAAGCGCCTG GCACAGTGTT GGGGACGCAG
    801 TAAATGCTCA AGGAATGATG ATTATGGATA CACCTATTAC ATATATGGTA
    851 AAATAACGCT TTATATCATC TGTCTCCTTT AGGATTTGGG GTGGAAGGCA
    901 GGCATGGTCA AACCCATTTC ACTGACAGGA GAGCAGAGAC AGGACGTGTC
    951 TCTCTCCACG TCTTCCAGCC AGTAAAAGAA GCCAAGCTGG AGCGCAAAGC
  1001 CAGGTGTTCT GACTCCCAGC GTGGGGGTCC CTGCACCAAC CATGAGCCGC
  1051 CTGCTCTGGA GGAAGGTGGC CGGCGCCACC GTCGGGCCAG GGCCGGTTCC
  1101 AGCTCCGGGG CGCTGGGTCT CCAGCTCCGT CCCCGCGTCC GACCCCAGCG
  1151 ACGGGCAGCG GCGGCGGCAG CAGCAGCAGC AGCAGCAGCA GCAGCAGCAA
  1201 CAGCAGCCTC AGCAGCCGCA AGTGCTATCC TCGGAGGGCG GGCAGCTCCG
  1251 GCACAACCCA TTGGACATCC AGATGCTCTC GAGAGGGCTG CACGAGCAAA
  1301 TCTTCGGGCA AGGAGGGGAG ATGCCTGGCG AGGCCGCGGT GCGCCGCAGC
  1351 GTCGAGCACC TGCAGAAGCA CGGGCTCTGG GGGCAGCCAG CCGTGCCCTT
  1401 GCCCGACGTG GAGCTGCGCC TGCCGCCCCT CTACGGGGAC AACCTGGACC
  1451 AGCACTTCCG CCTCCTGGCC CAGAAGCAGA GCCTGCCCTA CCTGGAGGCG
  1501 GCCAACTTGC TGTTGCAGGC CCAGCTGCCC CCGAAGCCCC CGGCTTGGGC
  1551 CTGGGCGGAG GGCTGGACCC GGTACGGCCC CGAGGGGGAG GCCGTACCCG
  1601 TGGCCATCCC CGAGGAGCGG GCCCTGGTGT TCGACGTGGA GGTCTGCTTG
  1651 GCAGAGGGAA CTTGCCCCAC ATTGGCGGTG GCCATATCCC CCTCGGCCTG
  1701 GTAAGTAGGG GCAGGGTTGG GGACATAAGC AGGCATGGGG GCCCAGCTTA
  1751 ATAGTTTGTT TCAGTGAACA TTTTCTGAGG TCCTGTTACG GGCTGGGTGC
  1801 TCACGTAGGG AGCGCTGATG TGTTGAATTA GGACTAGACC CCTGTTTATG
  1851 TGGGACTCAC TTTCTGGTGG GAAGATCACA GGCAGTAAGC AAATACCCAA
  1901 GTAAATGTCA GGCAGTAAAG GCCACGCAGA GAATCACAGT AGAGCGCTGT
  1951 ACATGAGACC TTCGGGAGGC CACTTAAGAT CACGGTGATT TGGTGCCTTT
  2001 ACCCCCTCTC CTAATAGCGT CATGAGAAGT TAGTCTGAAA AGTCATTTGA
  2051 ACAGTGTTTC TATTTGGGGA GCTATTAATT ATTTTGGGCG GTAGAAAGCT
  2101 CCCTTTTGTG GGACTGTCGC AGGCAGTATA GGACATTTAG CATCCCCAGC
  2151 CTTTCCCATA AACGCCAGAC CAACACCCCC CCGCCCCCCT GCCCCCGCCG
  2201 GCAACGTTTC CAGACGCCCC CTTGAGGTGG CATCTGGTTG ACCACCCCTA
  2251 GTTGAGAAAC ATTGCTTCCT TCCCCCAGCC TTCCAAGCAG GCATTTTGGT
  2301 CCCAAACAAG TATATCCAAT CTCTCTTTTC TTTTTAAATA ACTTTCTAAG
  2351 TGCTACCCAA GTTTCTTTTT CAAACAATGA TGGCAGTACT GTTTCTCCCC
  2401 TTTTTTTATT CTTCATTCCA GGATTAAAAT ACTATTTACA ACCTTAATGC
  2451 TTTCAGGCAT GGCCAGCAAA AAAGTTGGCA GTTTCTTTAT TCCTATTGGA
  2501 AGCTACATCT TTGTAAAGAA AGCTGCGAAA TGTTAAATAT GCAGTTGAAA
  2551 ATGGTGAAAA CATGGCTAAA TAGATAAGGT AGGCATTAAT GGCTGAAAAG
  2601 AGCAAAACTA GATGATTCTG CATTGATTGA GTTCCAGTTA CAATGAGAAT
  2651 CACACTACTT AGAATATGTA ACTTGATGGT CAAAGTAAAG GGGAATATCG
  2701 GCCATCATTT GAAAAGATAA AGTAGGCTTT GGTGGCTGAA AGAAAATTAG
  2751 GAAACCAGTG ACAAGAAAGA TTTGTTTTTT GATCTGTCGG TCATTTTAGG
  2801 CCAAATTACC TCAAGTCCCC TTTTCTTTTC TCTTTCTCCT TCTTTCTCTC
  2851 TTTTTACCTC TCCTTTCCCT CCCTGTCCTT CCCTGCTCTG CCCTCATTCT
  2901 CATTCCATTC TTGCCAGTGG TACTCGGGGC ATTGCTTAGT TGACCTGATG
  2951 GCAGAAGTCA CTGTTAAGGC CTGGGCTCAT GCTGGGACCT TCCTCCTGGG
  3001 AGTCTGACTG GTGGGTGGGG GTGGGTGCCA CATGGTGCCC TAATAGCGGT
  3051 CCACTTTGAA CCTGGGCATG CCCCTGCCCC TTAGCTGAGT AACATTAGGT
  3101 ACCTGACCAG CCCACAATTT ACAATGGGAG GAGAAGCGGT AGTCAGCTAT
  3151 GAGCCTCCCA CAGGGCAGCT TCTTCCCAAA GGGTGTTGGT AAGGGCTTCG
  3201 GCCATCAGGC TAGAGGGACG TCTCTCTGGC CATCAGCATT TTTCTAAGAT
  3251 TCACAGTAAA ACTAGTATTA ATGGCATGGA TCCCTACTCA TCTTAAATTT
  3301 GGCTTGTTTC TTTTTAATCA CTAGTTTATA ATATGGCTTC ATGCACAGCT
  3351 GCAGAGCTGC ATCTTGACAC CAGTGTGGCT TTTTACTGTA ACCAAAGTTC
  3401 CTGTTACCAC CATGGCCTCA AAGATTTGGC ATTCTTTAGC CTTTTTGTCT
  3451 GCGTTGTTTT AAGGGCTTTG ACATGCTGAA TTAAAATGTG GGGGGGTGGG
  3501 GATTTCTTTC AGTCCCTTGG CTTATTTTCA CCATTTGGAG TATGAGTTCG
  3551 ATTTTGTCAG GTTTAAAACT AGGAACCTCT TTTTGCTTTC TCTTTGAAAG
  3601 AAGTTAGTTT TATGTGTGTT GAATCTGTTG AGGCAGATAC TCCCTTTTTC
  3651 CCTTCCATAA AGGTTGCAAG GAGCTCCTTC GCAGCTGTGT TGTCCACACG
  3701 TGGCCTCGTC ACTCACTTTG ATGCTGAGTG GGCCTTGATT GTTTAGAATA
  3751 ATCTGTGGCT TGCAACAGGC ATTTCCTCAG TGGCCATTCC CCTACACCTA
  3801 GCCTTGTGGA TCTTGAGCAA ACTGCAGCCT TTTCCTGAAT CAGTGTCGGG
  3851 CCCCCAACAG GCAGCACTCA TCCCCTATCC CTCCCACCCC AACCCTGTCA
  3901 CATACACATA CATTTTCTCA TTCTGGCACT TTCCCTGGTT CTCACTGAGG
  3951 GTGGTTGCTT CTCCAAGGTG TGTGATTTGC TCTTTGTCCC CCAGAATCTT
  4001 TTCAGCCGTG AGATGATTCA TCCTGTACAT GTGTGCAGCA GCATTTGTCA
  4051 TTTTTTTTTT TTTTGCCAAT TCAATTAAAT CTCCACCTTG GGTTCTGTTA
  4101 TTGTCTATCT CCTTTACTAG TACTTTGAAC AGTAGCTGGT TTGTGCCTGT
  4151 AGACGTGAGG GGTTGATAAT GTTCATAAAA CCTCAGAGCT AGATGCAGAC
  4201 TCAGTGAACG CTGGGCCTAG CAAACACCTT GATAGCCCAG GCTGTAATAG
  4251 AATACCTGCA CGTAGGTCTA ATAGCCCAGT AGTTCCATTT TTATGTGCAG
  4301 AAGTTTAAAG AAGCTTTTGT AGCTCTTGCC CGCCAGCACA CACACCCACC
  4351 CTGCCACACC TGACCTGTAG CTGTTTGAGT TAGGAGCACC CTTTGGTCTC
  4401 ACTTGTGTCC CCAGCTGCCA ATGCACCATC TGGCATGTGG CGGTAGGTGT
  4451 GCAGTGGTTG TTGTGGAGTG GAAGTTTAAT GTCTCCATGG TGAACCTGCC
  4501 TGCCTCTCAC CTCCCTCAGG TATTCCTGGT GCAGCCAGCG GCTGGTGGAA
  4551 GAGCGTTACT CTTGGACCAG CCAGCTGTCG CCGGCTGACC TCATCCCCCT
  4601 GGAGGTCCCT ACTGGTGCCA GCAGCCCCAC CCAGAGAGAC TGGCAGGAGC
  4651 AGTTAGTGGT GGGGCACAAT GTTTCCTTTG ACCGAGCTCA TATCAGGGAG
  4701 CAGTACCTGA TCCAGGTAAG GTTCCTGGGG CCAACTGCAG GTTCTGGCAT
  4751 GGGATGGGCC AGGAGCCCTA ATCTCAGTGG TTAGGGGAGG TACTCCTTTC
  4801 CTGGCACGTG TCTCTGTTGC CTTTGCTGAA GCCGCAAGGC GCATCTGTTG
  4851 ACCAGCTGTG CCTCTGGTCT CTGTGCCTAG CTGTTGTATG TCCCCGGGAA
  4901 AGCCTGGTAT AGGACCTAAG TTGTCACAAA GTAATAATGG CCTTCGTCTC
  4951 TGTGGCATTT TAGAGCTTAG CATGGGTCTT GAAGGTTTTG AGCCACAGCC
  5001 TGGGCTCACT TCCTGCCTTA ACCACCGATG ACTACTGTGA GCGCCTTAAC
  5051 ATCTCTAAGT CTTAGTTTCC TTTTTTATAA AAAGGCAGAC ATAACAGAAA
  5101 TCTCATAGGA TTAATAGGAG GGTTGGAACA ATGCCTGCAT GTCAAACACT
  5151 CAGCACTCTG CCTGGTGTAT AGTAGTGGCA ATTCTTAATT TTATGAAAAG
  5201 TGTTTTTTCA CTGGATCTTC ACAACAGCCC TAGAAGATAG GCCAGGCAGG
  5251 GGAGAGCAAC CTTACCCTAT AGCTGAGGGT GCTGAGGCTC AGACAGCCTT
  5301 GTTGACATGC TCAGGGCCAC AGAGCTTTTG AGTGGCAGGG TTGGGGCCAG
  5351 ACCAGATAGC CCTGAAGGCT TTATTTTGGC CACTCTGTAT CTACGTTGCT
  5401 CAGAGCTATT GTTGGAAGCT GAGAAGGACT TGCACATTGG GATTGAGCCA
  5451 GGCCTGCATC TTAAAGGGTG GCTAGGATTT GGGAAGGCAG GCCCCTTACA
  5501 GGTGATGGGG CAAGCATGAA CAAGCATGAG GATTCTGTAT TTGGTGTTGA
  5551 AGGCTGTGTG CTGGGAGGGG AGGCTGTTTG AGGAGCTGAG GTGGGGCTGG
  5601 AGGTCCACAC CACCAAGCAG TGGTGGGCTG GCCCCACAGT TGCAGCCTCC
  5651 CTCCTTCCCT TCCCTTTTCT CCTCCTCCTC CTCAGGGTTC CCGCATGCGT
  5701 TTCCTGGACA CCATGAGCAT GCACATGGCC ATCTCAGGGC TAAGCAGCTT
  5751 CCAGCGCAGT CTGTGGATAG CAGCCAAGCA GGGCAAACAC AAGGTCCAGC
  5801 CCCCCACAAA GCAAGGCCAG AAGTCCCAGA GGAAAGCCAG AAGAGGCCCA
  5851 GCGGTGAGAG CACACTGCCG GTGGGCAGGA GCATAGTGCT TGGGACCCCC
  5901 TCTCACCAGC CCGTCTGGCC CGAGGCCAGG CTGATCTGCC ATGTCCCTTG
  5951 CTCTGGTTCC CCAGATCTCA TCCTGGGACT GGCTGGACAT CAGCAGTGTC
  6001 AACAGTCTGG CAGAGGTGCA CAGACTTTAT GTAGGGGGGC CTCCCTTAGA
  6051 GAAGGAGCCT CGAGAACTGT TTGTGAAGGG CACCATGAAG GACATTCGTG
  6101 AGAACTTCCA GGTATGGTGC TGGAGGGGGC TCTGGGGACA TGGGCTGTGG
  6151 CACACCCCTA GCTGCACTTG GGGAGATGCA GCTGCCAGGC CTGACCCTGA
  6201 GAGCTGGTGG TGGTAATGGG ATGGCTGCCC ACCTTGCGCC TTCCTGTCAC
  6251 CTTGTGCCAG GACCTGATGC AGTACTGTGC CCAGGACGTG TGGGCCACCC
  6301 ATGAGGTTTT CCAGCAGCAG CTACCGCTCT TCTTGGAGAG GTGAGGGGGA
  6351 GCCCATGTGG GAATCTCTGG GGGTCAGTGT GTTCCTGGTA CCCGGGCCCA
  6401 CTGTAATCAG GTGGCGCTGG TTCTATCTCA GGTTGGGGAC CTTAGCTTTT
  6451 CTAGGCTGAA AGAATGGAGC CCTTCTGTTC AGTGGTGTCC ATCTGGGCCC
  6501 TGGACTCTGG ATTTGACAGA GGCCCTGAAG GGGAGGGCCA TGGAGTTGTG
  6551 CTTGTGTGTC ATGTGCACGG TCCTGGTTTA CTGTGCACCT TCTCTAACTA
  6601 GATCCTTAGC CAAGGGCTTC ACATACAGCG TGGTTATGTT TATTAATGAG
  6651 TCTGTCTTAT GAAGTGACCC TTGTATGCTG AAAATTCAGG TATATTTGTA
  6701 CCAAAGATAT GGAAAGAAAA AAGAAGGGAG GAAAATTTGG GTGTAACTTT
  6751 TGACTCCCTC AGAGCTTAAC TACTAATAGC TTGCTGTTGG CTAGAAGCTT
  6801 TACTGATAAC ATAATACATA TTTTTTATGT TATACGTATT ATATACTGTA
  6851 TTCTTAAAGT AAGCTAGATA AAAGAAAATG TATTAAGAAA ATCATGAGGA
  6901 GAAAATATGT TTACTATTCA TTAGGTGGAA GTGGATCATC ATAAAGATAT
  6951 CTATCCTTCA CGTTGAGTAG GCTGAGGGCG GGGGTTGGGC TTGCTGTCTC
  7001 GGGTGGCTAA GGCTGAAGAA AATAAATGTG TAAGTGAACT TGCACGATCC
  7051 AGACATGTGT TGTTTAAATG TCAGCTGTAT TTTACCACCC AAGTTGTGAG
  7101 GTTCAGGCAT GATGTTTTTC ATGTATGGGA TTATTAGCAC AGTGCCTGGC
  7151 ACAGAGTCAT TACTCCACGT GTGGCAGCCA TTTTCACTTT TCCCATCTAT
  7201 ATTTCCCACA TTACCCCTGA GGATGGGATG ATATTGTTCC CATTTTATAG
  7251 ATGAAAGAAC TGAGGCTCCG AGAGATGGGG TTGCTTACCC AGGGATGAGT
  7301 AACAGTAGAG CTGGGATTTA ATGCCGTCTG ACTTTTGAGC TGTGCCATGT
  7351 CAGTGGCTGG GTTGAGGCTT GCTAAACCAG CTCAGGGATT GGGCCAGTCT
  7401 TGCCTCCTGT GGTCATTTAT GGCAGCTCCT GGTGTTTGCC TCCAAGGTGT
  7451 CCCCACCCAG TGACTCTGGC CGGCATGCTG GAGATGGGTG TCTCCTACCT
  7501 GCCTGTCAAC CAGAACTGGG AGCGTTACCT GGCAGAGGCA CAGGGCACTT
  7551 ATGAGGAGCT CCAGCGGGAG ATGAAGAAGT CGTTGATGGA TCTGGCCAAT
  7601 GATGCCTGCC AGCTGCTCTC AGGAGAGAGG TAGCCAGGCC TTGGGTGGGC
  7651 AGGATCTAGG CAGGGGACTG GCAGGTGGGC GGCCTAGCCT TCGGCTTAGC
  7701 CTTAGCCCTG CCCTAGTGGA CTGGCTCTGT AGGTACAAAG AAGACCCCTG
  7751 GCTCTGGGAC CTGGAGTGGG ACCTGCAAGA ATTTAAGCAG AAGAAAGCTA
  7801 AGAAGGTGAA GAAGGAACCA GCCACAGCCA GCAAGTTGCC CATCGAGGGG
  7851 GCTGGGGCCC CTGGTGATCC CATGGATCAG GAAGGTGGGG AGCATGGGTG
  7901 GGAGGTAGGG TAGGGTAGGG GTTGTCTCTG GGAAGGTCCT GTGATTGAGG
  7951 GGGTCCTTCG AAAGGATTGC TCCAGCCTTC TGGAGATGAG CGGGTGGGAG
  8001 CAGATCTTAT TGAGAGTTCC TTCTCCTGCT CCTGATTGTC TTCCCCCACC
  8051 CTCACAGACC TCGGCCCCTG CAGTGAGGAG GAGGAGTTTC AACAAGATGT
  8101 CATGGCCCGC GCCTGCTTGC AGAAGCTGAA GGGGACCACA GAGCTCCTGC
  8151 CCAAGCGGCC CCAGCACCTT CCTGGACACC CTGGGTGAGC CCTGCCCACC
  8201 CCCAGCAGTG TATCTAGAGT CTACCCTTGC TCCATTCTCA GGACAGCCCT
  8251 GGTCTGGGTT CTGGCACAGA GGCATCATGC ACATGTATAC TTATTGACCT
  8301 GCTGCCATTC AGTCACACTG TCTTCCAGTC CTATTCTCAT TTGCTCACTC
  8351 TGGACCGGCT CACTGGACTC ATTCAGCACA GTGTTGTGAG CACCTGCTGT
  8401 GCAATGGCCC GTGGCAGCCA CCGGGTGTAC ACACTGGAGC ATAGCTCCTC
  8451 CTTTCCAGTA GTTCTTTTTC CTAGGAGGAG CCAGGCACGT AGACCAGCCA
  8501 GTGCAGCTAG TGTCCATAGG TAGAGTTCTG ACTCTGCCTC GGGAAATAAA
  8551 TCAAGAAGGC TTCCTTGAGA AGGTGCCCCT TCCTTTGAGC CTCATAGGGT
  8601 GGCAGAGATG AGAAAAAGGG CAGCCAGGGT GAGCAGCAGG GTGCCAGCTT
  8651 TGCACCTGCA AGACCCTGAG AGCAAGTGTC CTGAGTGCCT TGCTAGTCTC
  8701 ACCCTGGGCT CAACTCTGGT GAACAGCCTG CAAGAGAGCA CCCAGAAGGA
  8751 CTGGTGTTTC TCTAGAGGGG TGGGGAGGGC AGATCTGCTC CCTCCTCTGG
  8801 TCAGTTACCC TGGATGAAAT GGAGCTTGGG AAGGAGCCCT GCCCTGGGTC
  8851 AGGGTATGCT TTTGTGTCCT GGCTTCTGAC TAGTCCAGTG GGACTGACTT
  8901 AGTGTCTTTG CTTTTGAAAT ATTCTTCTAG AGGATTCCAT GGGGGTCCTG
  8951 GCTAAAGCAT CCCAGAGGAG GGGATGGCGG CTGTAGGCTG GGGTCACCAG
  9001 AAAGCCCCAG GGCTTTGGAG GGTGGGTGGG GACATTGTGA GAGAGAGAAC
  9051 CTTCCCCCCA ACAACTGCCC TTACCATCGT GACACTGCTG TCTTCCTGCT
  9101 GGGACGTAGA TGGTACCGGA AGCTCTGCCC CCGGCTAGAC GACCCTGCAT
  9151 GGACCCCGGG CCCCAGCCTC CTCAGCCTGC AGATGCGGGT CACACCTAAA
  9201 CTCATGGCAC TTACCTGGGA TGGCTTCCCT CTGCACTACT CAGAGCGTCA
  9251 TGGCTGGGGC TACTTGGTGC CTGGGCGGCG GGACAACCTG GCCAAGCTGC
  9301 CGACAGGTAC CACCCTGGAG TCAGCTGGGG TGGTCTGCCC CTACAGGTAA
  9351 GGCTTAGGCC CAGGGGAGGA AGGGGCTGGA GCCTAGGGAC CCCTTCCCCT
  9401 GGCTGGTCAG CTCAGGCTAG TGGAAAGAGT TTGGGTTCAA GAGTCTGGGT
  9451 TCAGAAGAAG GGAAAACAGG AAAAAAATTA ACACACACAC ACACACCCTC
  9501 TCTCTCTCTC TTTCTCTCTC TCTCACTCAC TCACTCACTC TCTCTCTCAC
  9551 TCACTCACTC TCTCACTCAC TCTCTCACTC ACTCACTCTC TCACTCACTC
  9601 TCTCACTCAC TCACTCTCTC ACTCTCTCAC TCACTCACTC ACTCACTCAC
  9651 TCTCTCACTC ACTCTCTCAC TCACTCACTC ACTCTCTCTC ACTCTCTCAC
  9701 TCACTCACTC TCTCACTCAC TCACTCACTC ACTCACTCTC TCACTCACTC
  9751 ACTCACTCTC TGGGTTCAGG TTTTTTCTTC CATGGCTACC CTTACCCTCT
  9801 GGATCTCAGA GCTCTGGGAG GGAGTATGTT GAGATGTTCA CAGTGGGGAG
  9851 GACTAAAGGC CCTACTCTTG GGCCCAGAAG CATAGCTGCC TTCACAGGAA
  9901 CATGCGGAGG GCTGTTACAA GTAGCAGGGA GATGGGCTTT TAAAAAAGTG
  9951 TGTGTATATA ATTTGAGTGA TAATTATGGG CCAAGCAGTG CTTCCCTTAT
10001 TTGTTCCCCA AGGAGTCCCA TGAGCTAGAA TGGTTATCCC CATGTTGTAG
10051 TTGACAAAGG CTTGGTTGAC TTAAGATCAC AGACCCTGAG CTTTAGGCAG
10101 GCAGGTGTTG GGGAGAAACT TACAGTGGCC CAGAATTAAG AGTCCTGGCT
10151 CTTCAGGGCA GCCTGAGTCT CTTATGGGGC CATGGGACCA AAGGGGATAA
10201 CACTGGCCTT GCTCCTTTGA GCCCGAGGGT AGGTGAGCGG ACAGGAGCCA
10251 GCCTGCAGCT GGGCCTTGGG TCCTGTCCTC CCGCTGCTGT GCTCTCAGAA
10301 CTTCTCTTGA GACGGCAGCT CTGTAGTGTA AGAGGAACTT GGATTTGAGT
10351 GAGACAAGGC CTTGAACCCC AGCCTGCTGC CAGGGTGCTG TCATTTTCAG
10401 TTTGTCAATC AATCCCTGTC TAAAACCCGG GAAAGTGCTA TCTGGTTCTG
10451 CCTCAGAGCT GATTCTGAGG ACTAAACAAA GGGAATTGTG GAAGGCACTA
10501 GCAAGCTGCC TGGCCCAGAG TGGGCATCTG GTAATCAGCG GCTGCTGCTG
10551 CTACTGTTCT CTGCCCAGAG CCATCGAGTC CCTGTACAGG AAGCACTGTC
10601 TCGAACAGGG GAAGCAGCAG CTGATGCCCC AGGAGGCCGG CCTGGCGGAG
10651 GAGTTCCTGC TCACTGACAA TAGTGCCATA TGGCAAACGG TGAGGGCAGG
10701 CTCTGAACCT GAGCTTTGGG GAGGGGAGGT CTCTGTATTC CACCCAGGGA
10751 AGGGGCAGCC TTTGGGTGGG AGGCTGGCAC TGGTGGCTCA CCCCAGACTG
10801 GCCTGCAGTG TCTGAGTACC ATGCAGGGAG GGGCTGGTGG ATTGGGGCCT
10851 ACCCAGTCCC CTGCTTCACT ACTTTGGTCC TTGGACTGCT CCAGGTAGAA
10901 GAACTGGATT ACTTAGAAGT GGAGGCTGAG GCCAAGATGG AGAACTTGCG
10951 AGCTGCAGTG CCAGGTCAAC CCCTAGCTCT GGTGAGCAGT GCGCCGGCTT
11001 GGGTTCTCTA GGTGGGTGCT GGGTGGAAAG GGCTTCCTCT TGCCCACCTA
11051 GTTCTTCCCA GCCAGAGTTC CCTAGGTCTT AAGGGGGTTG GAGATGCCAC
11101 CCTGCCCCTG GGAGGCCCCA CACGTGTTGG AGCAAGGAGA AAGCCTGGGT
11151 GAGACCTCAT GGCCATCTTG TCATTTCCCA GCTGATGACG ACAGTTTCAG
11201 GCCCTTTTCC CACCCCCTAC CCCATGGCCC TTGCTGAATG CAGGTGCTGG
11251 AGCAGGGCCT GATATAGGTG TGTGGCCCTC ACAGACTGCC CGTGGTGGCC
11301 CCAAGGACAC CCAGCCCAGC TATCACCATG GCAATGGACC TTACAACGAC
11351 GTGGACATCC CTGGCTGCTG GTTTTTCAAG CTGCCTCACA AGGTGTGTCC
11401 TGGGTCATGG CCTGTCCTGT GGTGTTTCCT CATTCTGCTC AAGGCCCACA
11451 GCAGGCCTTC AGAGTGACAC ACCTGAGACT TTCCTTTTTG TGGGAATGAC
11501 TAGTAGTGGG ACAGAGTGTG ATTTCAGGCA CATACTGTCA TCTCTCAGCT
11551 TTTGTTTTTC TAATGAAAGT CGGGTGGCAA GGGGCATGGT GGTGGAATTA
11601 AATGACATGG GGCACGTCGT ATGTTTGGTA CGACATCTGG TACGTGATAG
11651 GTTTTTCCGA TTTGTTATTA TGCAGGGAGC CAGGTTTGCT TGTGTCTGTG
11701 TGTCTTAGGG GGCATGTGTG TGCACGTGTG TGTGTGCGTG CGCGCGTGCG
11751 CGCGTGCGTG ATACAATCAG GGATTTGCCT CAGACTGCTG AGGTTCTGGG
11801 CTCAGTGTTG GGAGGAGTGC AGGTACTCAC GTTGGTTCCC CACCCAGGGG
11851 TCTGCCACCT GCCTCCAGGC CCTGCTTCCT TTGCTCTGTC CAGGATGGTA
11901 ATAGCTGTAA TGTGGGAAGC CCCTTTGCCA AGGACTTCCT GCCCAAGATG
11951 GAGGATGGCA CCCTGCAGGC TGGCCCAGGA GGTGCCAGTG GGCCCCGTGC
12001 TCTGGAAATC AACAAAATGA TTTCTTTCTG GAGGAACGCC CATAAACGTA
12051 TCAGGTGGGC CACCATGGGA GGAGTCCTGG GATGCCTTTC CCCTCTCTTC
12101 CCACCCAGGG ACCCCTGACT AACCCTGGAT TCCCACAGAG GGCCAGCCTG
12151 ACTATGGTCT AGAGGCCTGG CTACTTTTGG TCCTGGTGCC ATGGACCTTG
12201 GGCAGGTCTC CCCTGTAGCT TCAGTTTCCC TGTTAATGTA AAAAGAATGG
12251 TGCTGTAGGA CCATGAGAGC CCTTCGTAGC TCCAACAGAA CTTCTTGGTG
12301 TAACTGCTGG AGCCGTGGGC TATGGCTGAG GACCATGGAG AGCTGGTGGC
12351 CTGTAAGCCC TGTTGGGGGC TGGGAGCTGG GTCTTCTAGT CTGGAATGGC
12401 AAATGTATTC ATCTTGAAGG CCATTTCCAA GGTGGTTGTG GCCATCAGCA
12451 CACTGGCGAG CAGAGTGGGT GTTGGGATGG TGAAGTCTGC CTGTGTGTAG
12501 GAAGAGGCAT TGGTGGAAGG AGCGCCTCAT GGATGCCCCC CGGAGAGGAG
12551 CGGAAGCTCG CTCGGAGGCC TGGCCGGTTC CCAGATGGTT TATGCTCTTG
12601 ATTGGTGTAT CATAGGGCCC CAGTTCTTGG CTGAGCCAGG GCTCACCTTG
12651 AGTCCAGTTA GTGAGGCTGG GTAATGGAGT ATAGCAGTCC TGGAGGTGGG
12701 CAGGTGAGGG CCATGGTGGG ATGTGGGATA GATTCTGCTT CCCATGGCTG
12751 TGCTGAGCCT CACGTTGTCT GTCCCCACAG CTCCCAGATG GTGGTGTGGC
12801 TGCCCAGGTC AGCTCTGCCC CGTGCTGTGA TCAGGTATGG TCTGCTGAGT
12851 GGTTGTAGGG ATAGGAGAAC TGAGGTGAGG TGGTAGGTCC TAAGGCCAAA
12901 GCACCCTGCT AAGACCCATT TCCTTCCCCT GCACCCCACC AGGCACCCCG
12951 ACTATGATGA GGAAGGCCTC TATGGGGCCA TCCTGCCCCA AGTGGTGACT
13001 GCCGGCACCA TCACTCGCCG GGCTGTGGAG CCCACATGGC TCACCGCCAG
13051 CAATGCCCGG GTATGTGACC TCTGTACCTC TGGCCCCTGC TCTTCCTCTC
13101 CCAGGTCTGT AGAAACTGGG CTCTGAGGGC CTTTAGGTAT TTAGTGAGGA
13151 TCATGAAAAG GACCCTGTGA TCTGGGTCAG GCAGGACTCT AGTCAAATCT
13201 GGCTTCATGA TTTCTGTCCA CTCCTTCAGT AAATATGTTC TGGGCACCTG
13251 CTCCTGGCCA GACCGTGACA GGCGTAATAG CTACAGCTCT CATGGAATTT
13301 AGATAGGACC GTGTAGGTGA GGGGTCTGGC ATAGCGCTAG GCATAGAGTA
13351 GATTCTTTAC CTGTCACACC AATTGCTGAT AGGTGGCCAT CTCTGGAACT
13401 GTGGAATTTC AGCAGTGCTG TCTGGCATTC TCTAAAGCCA TCCCCTCAGG
13451 AAAGGCTCTA GCTCTTTCTC AGTCAACTCT GGCTCCAGGA ATGGGGTAGG
13501 AAGAGTCTCA TTTGGGTATC TCACTCTTCC CACAGCCTGA CCGAGTAGGC
13551 AGTGAGTTGA AAGCCATGGT GCAGGCCCCA CCTGGCTACA CCCTTGTGGG
13601 TGCTGATGTG GACTCCCAAG AGCTGTGGAT TGCAGCTGTG CTTGGAGACG
13651 CCCACTTTGC CGGCATGCAT GGTGAGCAGG AGCCGGGGTT GGGGCAGCCC
13701 AGCCCCTCAG CATATTGACA GTTCTGATGA ACATTGGGCA GAATGTTCCT
13751 GAGCTGCTTT TCTCACTCCT GCTTGTCTTC CAGGCTGCAC AGCCTTTGGG
13801 TGGATGACAC TGCAGGGCAG GAAGAGCAGG GGCACTGATC TACACAGTAA
13851 GACAGCCACT ACTGTGGGCA TCAGCCGTGA GCATGCCAAA ATCTTCAACT
13901 ACGGCCGCAT CTATGGTGCT GGGCAGCCCT TTGCTGAGCG CTTACTAATG
13951 CAGTTTAACC ACCGGCTCAC ACAGCAGGAG GCAGCTGAGA AGGCCCAGCA
14001 GATGTACGCT GCCACCAAGG GCCTCCGCTG GTGAGGGTCC CTCTCCCATC
14051 CACTTTAACA CCCAGGACCC GAGGCCTGCT TTACTGTCCT TTAGTACTAC
14101 CATCTGTTCT ATCTCCTGCC CATTACTTGA ACTCTCACCT AGCCCCTCTC
14151 CTTCCACACC TGTGTAACCT GGTTCCAGGA TGATTTGTCC TATTGTGACA
14201 TTTGGTTGCT TTATAGTCAG CCTTAAACAG TTTTTCCTCA TGGGAGTAAA
14251 GCTATACTTT TGGTATACTG TTACCAAGTG GTAGCATCTT GACAATTCTG
14301 ATTATGCTGC ATAATGAATA ATACAGGGGT TGCAAACTCA GATGCCTACA
14351 GGGAATGAGA GCAAATGGAG TGGGTGGAAG ACAGGAGTTG ACAGGAGGGC
14401 GCTGTGGCAA ACTGGAGCAT GTAGGCTGAT GTTGATACTG GAGAAAGCAT
14451 TACCAGGCCT CCAGGTTACT TAGCCTAGCT CTCCAATTTG TTTCCTCTGA
14501 TCGTACTGCA TACTGTGTGC TCAGGGCCTT AGCAGACTCT CTGCAGGGTT
14551 CCAAAAACAT TGAGGGAAGA GAGGTACAAC TTCCTGAGGT ACAGTACACT
14601 GTCCACATTT AATTAGCTGG CTCATTGTGG AAACTTCACT TTCTCGTCAA
14651 CAACTAAAAG TTAAGTATGT GATAAATGAT ATAGTGGTTG ATGACTATAA
14701 ATGCAGGGAA GGGGAGCTGA GTATCGTCCA GTGGATAAAG TGAGGTCGGG
14751 TAAGGCTCAT ACCGTGAGCA GCGTGTGCTG GTGGAGGCGA GAAAGGTGGT
14801 GGGGCTTTAG TTGTGGACAC CTTTGAAAGT GTCACAGGAG TTTGGACTGT
14851 GGGTGCAGGT GGTGGGGAAG CCATTTATGC GAGTGACGTG TCTCTGGAGC
14901 CTTCAGGCGA CAAGCCTTGT GAGGTCTGCA GGTTAGATGG AAGCTGGGAG
14951 TTGTCTAGGG TTGTGGCAGT TGAGAGGGGT AAGCCAGGCC TGGCTGTTGT
15001 GTTTTCTGCT TCAACAAATG CCCCCTCCCC TTCAGGGAGT AGCCTATTCT
15051 TACCCCTATC CCCCCAAATC TAGAGTGATG GCCCTTGCTG CCTCCTGAAT
15101 AAAAGGCCCG TGTTGGTCAT TGGGCAATTC AGTGTCTAAA GAAACAGGAC
15151 AGTAGGAATA GTGGTGCCTC CTGTGCTGGA GTCTTTGTCC TTTATTGGGC
15201 TACCATGGGG TGGCCCAGGC TTTGGGGCTA CAAAAGCCTG GGCTGCATCT
15251 CTTTCTAGCT CCATGATCCT AGGCAAGGCA CTTAGCCTCT CTGAGCCGTT
15301 TCTTCCTCTG AATAAAAGCC TTTAGGGGAC TGGCATGATG TCAGTGTTTT
15351 TAAAAGTTGA AGTGATATGT GAACATTCCT TGCCAAGGCA CTAGCGTGGC
15401 ACAGGAAGCA CTCCCGTGGA ATGATGGTGA TAACACTGCC CCCAGGTATC
15451 GGCTGTCGGA TGAGGGCGAG TGGCTGGTGA GGGAGTTGAA CCTCCCAGTG
15501 GACAGGACTG AGGGTGGCTG GATTTCCCTG CAGGATCTGC GCAAGGTCCA
15551 GAGAGAAACT GCAAGGAAGT AAGAACCTTC TTTGTGTTAA GGATGGAGGG
15601 AGGGGTCTGG GCTTGCCCCA GAAGAGCTTG GATGCTTTGT TTTTTAGCTT
15651 TGAGATGCTG AAAGACAAAG TCTGCCCTCT GTTTCTGGTC CCTTAGGTCA
15701 CAGTGGAAGA AGTGGGAGGT GGTTGCTGAA CGGGCATGGA AGGGGGGCAC
15751 AGAGTCAGAA ATGTTCAATA AGCTTGAGAG CATTGCTACG TCTGACATAC
15801 CACGTACCCC GGTGCTGGGC TGCTGCATCA GCCGAGCCCT GGAGCCCTCG
15851 GCTGTCCAGG AAGAGGTATC TTGCTACCTT TGGAGCATGG GCAGAGGGGC
15901 CCCAGGGAGG GCAGGGCAGA GCTCCCTGTG GACCTTACCA ATGTTTGTAG
15951 GTAGGGCCAG AGTGAAGCTT CTCTTGGGGC TTCTACCCTG GAGTTAATTG
16001 GTATGTAGCA TAGCCCCTTT CACCTCTGCC CACCTTCCCT TCCCAGTTTA
16051 TGACCAGCCG TGTGAATTGG GTGGTACAGA GCTCTGCTGT TGACTACTTA
16101 CACCTCATGC TTGTGGCCAT GAAGTGGCTG TTTGAAGAGT TTGCCATAGA
16151 TGGGCGCTTC TGCATCAGCA TCCATGACGA GGTTCGCTAC CTGGTGCGGG
16201 AGGAGGACCG CTACCGCGCT GCCCTGGCCT TGCAGATCAC CAACCTCTTG
16251 ACCAGGTATG CGGGGCCCAT GGCCTCTAGC CTGGCCATGT GCTCCTATGT
16301 GGGGCTTTGG GTGAGCGTTC CTTGGGCCAG ACTGGTCAGT TTTGACTTTT
16351 CATCCCCCTA GAAGTGAATG TTTCAGCTTA TTTATTTATT TCTAATTTTT
16401 AAAAAGTTGT AGAAGTCCTA AAAAGACTAG CCTCAATTCG TAAAAAAAGA
16451 GTTATTGGGT TTGAAAATGT GAAATACCAA GACTGATCAT TGAGGGAAGC
16501 AGTGAGGTTA GGGGAATTGT TCCGAAGGGT GGTACTCACG CTTTTCTATT
16551 TGGAAAATCA AATGACAGAA GCCTTTTCTC ATTTCATAGA AAATTGAGAT
16601 GTTTGTTTTT CTTTCTCCCA TAAATGTTTT CTTTCTTAAG TAAGTGCCAA
16651 AAGTTTGTTA TTTGACTGCT AACAGAAAAC ACTGTTAATG GGGACACTCA
16701 AATGTGATTT TTAAAAATAT CTTATATATT TTATATATTG AGTTGTATTT
16751 TCTTGTAGTA AAATTCCTAG TTCATATGGA TGAATTAAAT ATTACCGTTC
16801 CATGTTGATC TGCCACTCAG AACCAGTTTG GGAACCATGA TCTATCCTGA
16851 TTATTGGGTA AATAACAGAT GTTTACAATA TTCAACATTG TTCCCATTGC
16901 CCTCTTAATC ATCATCTCCG GGAGGTTATG CTTAACAAAG CTAAAAGTCC
16951 TCATTTATGC TTCAAAGTCT GGCCCAATTG GAAGTGATTT CGTATATTAA
17001 TTAATAAAGT GTACCAAACT GGGAAAAAAA AAAAAAGTAT GTTGAGTCCA
17051 TAATTGCATT TCAGTATCTC AGTGGGAGGT TAGGCTGCTG GATGGAAAAC
17101 AGTGCTGGAC CTTCACCTTT CTTGACTTAG CTAAGTGAAC AGATGGGGTG
17151 TTGGTCCAGG GGAAGCCCTG CTCTAAGGGG TGTGGGGTCA TTGCTCCAGG
17201 AGTGATGCAT CTGTTCACAG GAGGGGCATG ACTGTGAGAG TAGATTGGGT
17251 CTCTTTCAGG TGCATGTTTG CCTACAAGCT GGGTCTGAAT GACTTGCCCC
17301 AGTCAGTCGG CTTTTTCAGT GCAGTCGATA TTGACCGGTG CCTCAGGAAG
17351 GAAGTGACCA TGGATTGTAA AACCCCTTCC AACCCAACTG GGATGGAAAG
17401 GAGATACGGG ATTCCCCAGG GTGAGCACAA CACATTTGTT CCTCATTACA
17451 CATAGGATCT GAGGTGGACT AGAAAGTGGG TCTTGGAGAA CAGGAAACTT
17501 GGGGCCCCAG AGAATCCACT CTTGACTCAG GCTATATTCT AGGCTAATTT
17551 CAGTTTATAA GGTGCCCTGT GTCCAGAGTG AATGTGATAT GATGTTTCAG
17601 AAATGAAGGC AGCAGAGCTT CAAATATTCT ACCTGTACCT GTCCCCTACT
17651 TCAACCACAG AAGAAATGTT TAAAGATAAT TTATTCTATA GAGTGCATTC
17701 TTGCACTCTA TAGGTGACAG AAAAACAAAC TGTGCTTTAA ATACCAAACA
17751 AGTAAATCAG AAAGCTTATT TTCTATTTAA AATATATCTA AGACACACTT
17801 ATATAAAAAG AAAAGAGACC CTCCTAACAT GTAACATTAC CGTTCGTGGC
17851 AATTGTTCTC AACCTTTCAC TCTCCTTTTG ACCTTAGCAT TAAGCTCCTT
17901 TGCTCACTTC TGAGCTCTCA GTTACAGTTC TTGAGGTGGC ATCCTAACCA
17951 ATTTGCACTA TCTTTCAGGT GAAGCGCTGG ATATTTACCA GATAATTGAA
18001 CTGACCAAAG GCTCCTTGGA AAAACGAAGC CAGCCTGGAC CATAGCACTG
18051 CCTGGAGGCT CTGTATTTGC TCCCGTGGAG CTTCATCGGG GTGGTGCAGG
18101 CTCCCAAACT CAGGCTTTCA GCTGTGCTTT TTGCAAAAGG GCTTGCCTAA
18151 GGCCAGCCAT TTTTCAGTAG CAGGACCTGC CAAGAAGATT CCTTCTAACT
18201 GAAGGTGCAG TTGAATTCAG TGGGTTCAGA ACCAAGATGC CAACATCGGT
18251 GTGGACTACA GGACAAGGGG CATTGTTGCT TGTTGGGTAA AAATGAAGCA
18301 GAAGCCCCAA AGTTCACATT AACTCAGGCA TTTCATTTAT TTTTTCCTTT
18351 TCTTCTTGGC TGGTTCTTTG TTCTGTCCCC CATGCTCTGA TGCAGTGCCC
18401 TAGAAGGGGA AAGAATTAAT GCTCTAACGT GATAAACCTG CTCCAAGGCA
18451 GTGGAAATAA AAAGAAGGAA AAAAAAGA
```

1. An in vitro method for diagnosis or prediction of Parkinson's disease in a subject, the method comprising testing for a mutation of a mitochondrial DNA polymerase POLG1 gene (SEQ ID No. 2) of the subject and, where the mutation is detected, diagnosing or predicting Parkinson's disease in the subject.

2. The method according to claim 1, wherein the mutation modifies the function of POLGL protein (SEQ ID No. 1).

3. The method according to claim 1, the method comprising testing for a plurality of said gene mutations.

4. The method according to claim 1, wherein the mutation is tested in a region extending carboxyterminally from the exoIII domain to the spacer and pol regions of the POLG1 protein.

5. The method according to claim 1, wherein the mutation is tested in a DNA location corresponding to nucleotide locations of SEQ ID No. 2 of 7554 to 18478.

6. The method according to claim 1, wherein the mutation is tested in at least one DNA location corresponding to an amino acid location of SEQ ID No. 1 selected from: 467, 468, 627, 953, 955, 1105 and 1236, or in at least one nucleotide location of SEQ ID No. 2 selected from: 8258-8259, 10873, 11381, 11818, 15661, 15686 and 17374.

7. The method according to claim 6, wherein the mutation corresponds to at least one amino acid change selected from: A467T, N468D, R627Q, R953C, Y955C, A1105T and Q1236H, or at least one nucleotide change selected from: 8258-8259 with an additional G, T10873C, C11381T, T11818C, A15661G, T15686C and C17374A.

8. The method according to claim 1, wherein the mutation is tested in at least one DNA location corresponding to an amino acid location of SEQ ID No. 1 selected from: 43-52, 312, 467, 468, 627,748, 953, 955, 1105, 1143, 1230 and 1236, or at least one nucleotide location of SEQ ID No. 2 selected from: 1168-1197, 8258-8259, 10873, 11381, 11818, 15661, 15686, 15790 and 17374.

9. The method according to claim 8, wherein the mutation corresponds to at least one amino acid change selected from: A467T, 43-52PolyQ43-50, W312R, N468D, R627Q, W748S, R953C, Y955C, A1105T, E1143G, S1230F and Q1236H, or at least one nucleotide change selected from: 1168-1197 with missing CAGCAG((cag)₈-allele), 8258-8259 with additional G, T10873C, C11381T, T11818C, A15661G, T15686C, G15790A and C17374A.

10. The method according to claim 1, wherein testing is carried out on a nucleic acid sample obtained from the patient.

11. The method according to claim 1, the method comprising testing for a mutant allele, wherein detection of at least one POLG1 mutant allele indicates that the patient has or is predisposed to having Parkinson's disease.

12. The method according to claim 1, the method comprising testing for a mutant allele, wherein determination that the subject is homozygous or compound heterozygous for a mutation indicates that the patient has or is predisposed to having Parkinson's disease.

13. The method according to claim 1, wherein said testing comprises a procedure selected from: allele specific oligonucleotide hybridization; size analysis; sequencing; hybridization; 5′ nuclease digestion; single-stranded conformation polymorphism; allele specific hybridization; primer specific extension; oligonucleotide ligation assay; temperature gradient electrophoresis; microarray; and mass spectrometry.

14. The method according to claim 13, wherein said size analysis is preceded by a restriction enzyme digestion.

15. The method according to claim 10, further comprising amplifying the nucleic acid sample.

16. The method according to claim 15, wherein amplifying the nucleic acid sample employs a primer pair selected from the sequences SEQ ID Nos. 3-11.

17. A diagnostic kit for diagnosis or prediction of Parkinson's disease in a subject, comprising means for testing for a mutation of the POLG1 gene of the subject and means for determining whether the mutation is indicative for Parkinson's disease.

18. The diagnostic kit according to claim 17, comprising means for amplifying DNA and a labeled polynucleotide comprising a nucleotide sequence complementary to at least part of the gene encoding POLG1 and containing at least one mutation.

19. (canceled)

20. (canceled)

21. (canceled)

22. An isolated polynucleotide comprising at least one mutation of the POLG1 gene, or a fragment of said polynucleotide.

23. The polynucleotide according to claim 22, being labeled.

24. The polynucleotide according to claim 22, wherein the mutation is situated in a region between the C-terminus and the exoIII domain of the POLG1 gene.

25. The polynucleotide according to claim 22, wherein the mutation is situated in a DNA location of SEQ ID No. 2 of 7554 to 18478.

26. The polynucleotide according to claim 22, wherein the mutation is situated in a DNA location corresponding to at least one amino acid location of the SEQ ID No. 1 selected from: 467, 468, 627, 953, 955, 1105 and 1236, or at least one nucleotide location of the SEQ ID No. 2 selected from: 8258-8259, 10873, 11381, 11818, 15661, 15686, 17374.

27. The polynucleotide according to claim 24, wherein the mutation corresponds to at least one amino acid change selected from: A467T, N468D, R627Q, R953C, Y955C, A1105T and Q1236H, or genomic nucleotide change selected from: 8258-8259 with an additional G, T10873C, C11381T, T11818C, A15661G, T15686C and C17374A.

28. The polynucleotide according to claim 22, wherein the mutation is situated in a DNA location corresponding to at least one amino acid location of SEQ ID No. 1 selected from: 43-52, 312, 467, 468, 627, 748, 953, 955, 1105, 1143, 1230 and 1236, or at least one nucleotide location of SEQ ID No. 2 selected from: 1168-1197, 8258-8259, 10873, 11381, 11818, 15661, 15686, 15790 and 17374.

29. The polynucleotide according to claim 28, wherein the mutation corresponds to at least one amino acid change selected from: A467T, 43-52PolyQ43-50, W312R, N468D, R627Q, W748S, R953C, Y955C, A1105T, E1143G, S1230F and Q1236H, or at least one nucleotide change selected from: 1168-1197 with missing CAGCAG((cag)₈-allele), 8258-8259 with additional G, T10873C, C11381T, T11818C, A15661G, T15686C, G15790A and C17374A.

30. A recombinant vector comprising the polynucleotide according to claim 22.

31. A cell line comprising the polynucleotide according to claim 22.

32. A transgenic non-human mammal or invertebrate comprising the polynucleotide according to claim 22.
